(19) United States
(12) Patent Application Publication
(10) Pub. No.: US 2017/0258751 A1
(43) Pub. Date: Sep. 14, 2017

(54) METHODS OF TREATING HEART FAILURE USING FATTY ACID FUMARATE DERIVATIVES

(71) Applicant: Vijaykumar Rajasekhar, Apple Valley, CA (US)

(72) Inventor: Vijaykumar Rajasekhar, Apple Valley, CA (US)

(21) Appl. No.: 15/457,908

(22) Filed: Mar. 13, 2017

Related U.S. Application Data
(60) Provisional application No. 62/307,474, filed on Mar. 12, 2016.

(51) Int. Cl. A61K 31/232

(52) U.S. Cl. CPC A61K 31/232

(57) ABSTRACT

Pharmaceutical compositions comprising Fatty Acid Fumarate Derivatives, and methods of using Fatty Acid Fumarate Derivatives and pharmaceutical compositions thereof for treating heart failure diseases, including heart failure with preserved ejection fraction (HFPEF), comprising the administration of an effective amount of a Fatty Acid Fumarate Derivative alone or in combination with statins (HMG-CoA reductase inhibitors) are disclosed.

### CROSS-REFERENCE TO RELATED APPLICATIONS

This application claims priority to U.S. Provisional Patent Application Ser. No. 62/307,474, entitled “Fatty Acid Fumarate Derivatives and Their Uses in Treating Heart Failure,” filed Mar. 12, 2016, the disclosure of which is incorporated by reference in its entirety.

### INCORPORATION BY REFERENCE

All publications and patent applications mentioned in this specification are herein incorporated by reference in their entireties, as if each individual publication or patent application was specifically and individually indicated to be incorporated by reference in its entirety.

### TECHNICAL FIELD

This disclosure relates to methods and compositions for treating or preventing a heart failure disease, including heart failure with preserved ejection fraction (HFPEF), in a subject in need thereof by administering to the subject a therapeutically effective amount of a Fatty acid fumarate derivative (FAFD) alone or in combination with one or more second agents useful for treating heart failure.

### BACKGROUND

Heart failure (HF) is major health problem in the United States (U.S.) and elsewhere. In the U.S., HF affects over 5 million people with approximately half a million new cases occurring each year. HF is the leading cause of hospitalizations in people over 65 years in age. HF has many potential causes and diverse clinical features. Symptoms of heart failure can include dyspnea during activity or at rest, cough, rapid weight gain, swelling in ankles, legs and abdomen, dizziness, fatigue and weakness, rapid or irregular heartbeats, nausea, palpitations, and chest pains.

About half of heart failure patients have heart failure with preserved ejection fraction (HFPEF). Distinct from traditional HF, i.e., heart failure with reduced ejection fraction (HFREF) in which the ventricle has difficulty pumping, patients with HFPEF show declined performance of the heart ventricle, not at the time of contraction, but during the phase of diastole. HFPEF patients show normal ejection fraction of blood pumped out of the ventricle, but the heart muscle does not quickly relax to allow efficient filling of blood returning from the body. Morbidity and mortality of HFPEF are similar to traditional HF; however, therapies that benefit traditional HF are not effective in treating or preventing HFPEF. Patients with HFPEF have an ejection fraction of ≧40%, ≧45%, or ≧50% depending on which definition is chosen from the literature. On the other hand, patients with HFREF have an ejection fraction of either ≦35% or ≦40% depending on which definition and guidelines are used. For ease of simplicity, and not to be limiting in any way, HFPEF may be considered as having an ejection fraction ≧40% and HFREF can be considered as having an ejection fraction ≦40%.

Other names for the two primary clinical subsets of HF are diastolic heart failure (DHF) and systolic heart failure (SHF). SHF, which is also known as heart failure with reduced ejection fraction (HFREF) involves an abnormality of the heart resulting in failure of the heart to pump blood at a rate needed for metabolizing tissues at rest and/or during exertion. DHF, which is also known as heart failure with preserved ejection fraction (HFPEF), is a clinical syndrome with symptoms and signs of HF, a preserved ejection fraction, and abnormal diastolic function. The clinical manifestations of IIFREF and IIFPEF have distinct differences in risk factors, patient characteristics, and pathophysiology. Moreover, medications proven effective in HFREF have not been found to be effective in HFPEF. At present there are no approved treatments for HFPEF.

In HFREF, medications such as beta-blockers, ace-inhibitors, angiotensin receptor blockers, isosorbide dinitrate, hydralazine, aldosterone inhibitors, and angiotensin receptor neprilysin inhibitors have been shown to provide benefit. However, these medications have not shown to be beneficial in patients with HFPEF, and are not approved therapies for HFPEF.

Given that there are currently no approved treatments to improve survival in HFPEF, there remains, therefore, a real need in the treatment of HFPEF for a product that can improve morbidity and mortality of patients with HFPEF.

The present disclosure addresses needs in subjects with HFPEF, as well as in subjects at risk of developing HFPEF, due to conditions including but not limited to hypertension, diabetes, COPD, atrial fibrillation, obesity, or ischemic heart disease.

Oily cold water fish, such as salmon, trout, herring, and tuna are the source of dietary marine omega-3 fatty acids, with eicosapentaenoic acid (EPA) and docosahexaenoic acid (DHA) being the key marine derived omega-3 fatty acids. Omega-3 fatty acids have previously been shown to improve insulin sensitivity and glucose tolerance in normoglycemic men and in obese individuals. Omega-3 fatty acids have also been shown to improve insulin resistance in obese and non-obese patients with an inflammatory phenotype, Lipid, glucose, and insulin metabolism have been shown to improve in overweight hypertensive subjects through treatment with omega-3 fatty acids. Omega-3 fatty acids (EPA/DHA) have also been shown to decrease triglycerides and to reduce the risk for sudden death caused by cardiac arrhythmias in addition to improve mortality in subjects at risk of a cardiovascular event. Omega-3 fatty acids have also been taken as dietary supplements, as treatment of dyslipidemia, and as an anti-inflammatory agent. A higher intake of omega-3 fatty acids lower levels of circulating TNF-α and IL-6, two of the cytokines that are markedly increased during inflammation processes (Chapkin et al, Prostaglandins, Leukot Essent Fatty Acids 2009, 81, p. 187-191; Duda et al, Cardiovasc Res 2009, 84, p. 33-41). In addition, a higher intake of omega-3 fatty acids has been shown to increase levels of the well-characterized anti-inflammatory cytokine IL-10 (Bradley et al, Obesity (Silver Spring) 2008, 16, p. 938-944). A recent study (Wang et al, Molecular Pharmaceutics 2010, 7, p. 2185-2193) has demonstrated that DHA could also induce the Nrf2 and the Nrf2-target gene Heme-oxygenase 1 (HO-1). This pathway could play a significant role in suppressing LPS-mediated inflammation.

Both DHA and EPA are characterized as long chain fatty acids (aliphatic portion between 12-22 carbons). Medium chain fatty acids are characterized as those having the aliphatic portion between 6-12 carbons. Lipoic acid is a medium chain fatty acid found naturally in the body. It plays many important roles such as a free radical scavenger, chelator to heavy metals and signal transduction mediator in various inflammatory and metabolic pathways, including the NF-kB pathway (Shay, K. P. et al. Biochim. Biophys. Acta 2009, 1790, 1149-1160). Lipoic acid has been found to be useful in the treatment of a number of chronic diseases that are associated with oxidative stress (for a review see Smith, A. R. et al Curr. Med. Chem. 2004, 11, p. 1135-46). Lipoic acid has now been evaluated in the clinic for the treatment of diabetes (Morcos, M. et al Diabetes Res. Clin. Pract. 2001, 52, p. 175-183) and diabetic neuropathy (Mijnhout, G. S, et al Neth. J. Med. 2010, 110, p. 158-162). Lipoic acid has also been found to be potentially useful in treating cardiovascular diseases (Ghibu, S. et al, J. Cardiovasc. Pharmacol. 2009,54, p. 391-8), Alzheimer's disease (Maczurek, A. et al, Adv. Drug Deliv. Rev. 2008,60, p. 1463-70) and multiple sclerosis (Yadav, V. Multiple Sclerosis 2005, 11, p. 159-65; Salinthone, S. et al, Endocr. Metab. Immune Disord. Drug Targets 2008, 8, p. 132-42).

O'Connell et. al, WO 2013/116194 have disclosed a method for treating or limiting development of heart failure with preserved ejection fraction (HFPEF), comprising administering to a subject, having or at risk of developing HFPEF, an effective amount of a pharmaceutical composition comprising docosahexaenoic acid (DHA) or pharmaceutically acceptable salts, esters, amides, epoxides, and prodrugs thereof, to treat or limit development of HFPEF.

Fumaric acid and its ester derivatives, either the mono alkyl hydrogen fumarates or dialkyl fumarates, have been used as therapeutic agents for the treatment of psoriasis (Joshi and Strebel, WO 1999/49858; U.S. Pat. No. 6,277,882; Mrowietz and Asadullah, Trends Mol Med 2005, 111(1), 43-48; and Yazdi and Mrowietz, Clinics Dermatology 2008,26,522-526); asthma and chronic obstructive pulmonary diseases (Joshi etal, WO 2005/023241 and U.S. 2007/0027076); cardiac insufficiency including left ventricular insufficiency, myocardial infarction and angina pectoris (Joshi et al, WO 2005/023241; Joshi et al, U.S. 2007/0027076); mitochondrial and neurodegenerative diseases such as Parkinson's disease, Alzheimer's disease, Huntington's disease, retinopathia pigmentosa and mitochondrial encephalomyopathy (Joshi and Strebel, WO 2002/055063, U.S. 2006/0205659, U.S. Pat. No. 6,509,376, U.S. Pat. No. 6,858,750, and U.S. Pat. No. 7,157,423); transplantation (Joshi and Strebel, WO 2002/055063, U.S. 2006/0205659, U.S. Pat. No. 6,359,003, U.S. Pat. No. 6,509,376, and U.S. Pat. No. 7,157,423; and Lehmann et al, Arch Dermatol Res 2002, 294, 399-404); autoimmune diseases (Joshi and Strebel, WO 2002/055063, U.S. Pat. No. 6,509,376, U.S. Pat. No. 7,157,423, and U.S. 2006/0205659) including multiple sclerosis (MS) (Joshi and Strebel, WO 1998/52549 and U.S. Pat. No. 6,436,992; Went and Lieberburg, U.S. 2008/0089896; Schimrigk et al, Eur J Neurology 2006, 13, 604-610; and Schilling et al., Clin Experimental Immunology 2006, 145,101-107); neurological disorders characterized by extensive demyelination and/or axonal loss including secondary progressive multiple sclerosis and Devic's disease (Gold, WO 2008/096271); ischemia and reperfusion injury (Joshi et al., U.S. 2007/0027076); AGE-induced genome damage (Heidland, WO 2005/027899); inflammatory bowel diseases such as Crohn's disease and ulcerative colitis; arthritis; others conditions (Nilsson et al., WO 2006/037342 and Nilsson and Muller, WO 2007/042034); and an autoimmune and Th1-mediated skin disease (Altmeyer et al, J. of the American Academy of Dermatology 1994, 30, p. 977-981).

In clinical studies with psoriasis patients that have been administered with fumarates, a reduction of peripheral CD4+ and CD8+-T lymphocytes has been observed. These agents have been reported to inhibit LPS-induced NF-κB activation in dendritic cells and endothelial cells in vitro (Loewe et al., J. Immunol. 2004, 168,4781-4787; Litjens et al., Eur. J. Immunol. 2004,34, 565-575). Dialkyl and monoalkyl fumarates have also demonstrated oral efficacy in the chronic experimental autoimmune encephalomyelitis (EAE) mouse model for multiple sclerosis (MS). In this particular model, C57BL/6 mice were challenged with the immunopeptide MOG 35-55 in order to induce disabilities that were equivalent to those exhibited by MS patients. Oral treatment with either dialkyl or monoalkyl fumarate resulted in a significant improvement in the disability score. The anti-inflammatory cytokine IL-10 was particularly elevated in the blood among the animals treated with either dialkyl or monoalkyl fumarate. Furthermore, histological analysis of the spinal cord of animals treated with either dialkyl or monoalkyl fumarate showed a strongly reduced macrophage inflammation (Schilling et al., Clinical and Experimental Immunology 2006, 145, 101-107). Dialkyl and monoalkyl fumarate esters have also been used in a number of reported studies with patients exhibiting the relapsing-remitting form of multiple sclerosis. Patients treated with 720 mg of fumarate esters daily for 70 weeks exhibited a significant reduction in inflammatory brain lesions, as noted by the reduction of new gadolinium-enhancing (Gd+) lesions in various MRI taken during the course of the treatment (Schimrigk et al., Eur. J. Neurology 2006,13,604-610). More recently, fumarates have been shown to activate Nrf2, a transcription factor that is responsible for the induction of a number of important antioxidants and detoxification enzymes that protect mammalian cells against reactive oxygen/nitrogen species and electrophiles (Lukashev, M. E. “Nrf2 screening assays and related methods and compositions” WO 08097596 A2; Wilms et al, Journal of Neuroinflammation 2010, 7:30).

Chronic oxidative stress and inflammation have now been linked to the development and progression of a number of debilitating diseases beyond multiple sclerosis. Activation of the Nrf2 pathway in order to resolve this chronic oxidative stress and inflammation appears to be a particularly promising new therapeutic target (For a review see Gozzelino, R. et al Annu. Rev. Pharmacol. Toxicol. 2010, 50, p. 323-54). For instance, small molecule activators of Nrf2 have now been shown to be effective in the cisplatin-induced nephrotoxicity mouse model (Aleksunes et al, J. Pharmacology & Experimental Therapeutics 2010, 335, p. 2-12), the transgenic Tg19959 mouse model of Alzheimer's disease (Dumont et al, J. Neurochem. 2009, 109, p. 502-12), the mouse model for COPD (Sussan, T. E. et al Proc. Natl. Acad. Sci. USA 2009, 106, p. 250-5), and the murine 4T1 breast tumor model (Ling, X. et al Cancer Res. 2007, 67, p. 4210-8).

FUMADERM®, an enteric coated tablet containing a salt mixture of monoethyl fumarate and dimethyl fumarate (DMF) which is rapidly hydrolyzed to monomethyl fumarate, regarded as the main bioactive metabolite, was approved in Germany in 1994 for the treatment of psoriasis. FUMADERM® is dosed three times daily (TID) with 1-2 grams/day administered for the treatment of psoriasis. FUMADERM(r) exhibits a high degree of interpatient variability with respect to drug absorption and food strongly reduces bioavailability. Absorption is thought to occur in the small intestine with peak levels achieved 5-6 hours after oral administration. Significant side effects occur in 70-90% of patients (Brewer and Rogers, Clin Expt'l Dermatology 2007, 32,246-49; and Hoefhagel et al., Br J Dermatology 2003, 149, 363-369). Side effects of current FAE therapy include gastrointestinal upset including nausea, vomiting, diarrhea and/or transient flushing of the skin. Also DMF exhibits poor aqueous solubility.

Fumaric acid derivatives ((Joshi and Strebel, WO 2002/055063, U.S. 2006/0205659, and U.S. Pat. No. 7,157,423 (amide compounds and protein-fumarate conjugates); Joshi et al, WO 2002/055066 and Joshi and Strebel, U.S. Pat. No. 6,355,676 (mono and dialkyl esters); Joshi and Strebel, WO 2003/087174 (carbocyclic and oxacarbocylic compounds); Joshi et al, WO 2006/122652 (thiosuccinates); Joshi et al, U.S. 2008/0233185 (dialkyl and diaryl esters) and Nilsson et al, U.S. 2008/0004344 (salts)) have been developed in an effort to overcome the deficiencies of current FAE (Fumaric acid ester) therapy. Controlled release pharmaceutical compositions comprising fumaric acid esters are disclosed by Nilsson and Muller, WO 2007/042034. Glycolamide ester prodrugs are described by Nielsen and Bundgaard, J Pharm Sci 1988, 77(4), 285-298.

Prodrugs of monomethyl fumarate and therapeutic uses thereof are disclosed in U.S. Patent Publication US 2010/0048651 published Feb. 25,2010.

Other prodrugs of monomethyl fumarate and therapeutic uses thereof are disclosed in US Patent Publication U.S. 2013/0203753 published Aug. 8, 2013.

Joshi et al, U.S. Patent Application Publication No. 2004/0054001, discloses using fumaric acid derivatives, such as monoalkyl and dialkyl fumarates, for treating cancers such as mamma carcinoma, colon carcinoma, melanoma, primary liver cell carcinoma, adenocarcinoma, kaposi's sarcoma, prostate carcinoma, leukaemia such as acute myeloid leukaemia, multiple myeloma (plasmocytoma), Burkitt lymphoma and Castleman tumors.

Kahrs U.S. 2013/0172391 discloses the use of MMF and DMF for the treatment of diseases including, among others, chronic lymphocytic leukemia.

Wustrow et al. U.S. 2014/0057918 discloses the use of MMF and prodrugs of MMF to treat a tumor.

NRF2 deficiency, demonstrated by NRF2 knockout in murine models, results in an earlier onset of cardiac dysfunction induced by pressure and volume overload (Li et al Arterioscler Thromb Vase Biol. 2009,29(11), 1843-50). Certain NRF2 activators such as sulforaphane, curcumin, carbobenzoxy-Leu-Leu (MG132), resveratrol, garlic organosulfur compounds, allicin, 4-hydroxynonenal (4-HNE), α-lipoic acid, hydrogen sulfate, and 17α estradiol have been used as therapeutic targets to reduce cardiac remodeling, but fatty acid fumarate derivatives have not been used yet to reduce cardiac remodeling (Zhou et al; J Appl Physiol. 2015, 119(8), 944-951).

Fumarates are cardioprotective in acute situations via activation of the NRF2 pathway in acute ischemia due to myocardial infarction (Ashrafian et. al; Cell Metab. 2012, 15(3), 361-71). However, Ashrafian et. al claims that fumarates are harmful in chronic situations, including heart failure.

Therefore, it is intended that the ability to provide the effects of fatty acids and fumarates in a synergistic way would provide benefits in treating heart failure diseases, including heart failure with preserved ejection fraction (HFPEF).

### SUMMARY

The present disclosure relates to methods and compositions useful in the treatment of heart failure diseases. The methods and compositions described herein comprise one or more FAFDs for the treatment of a heart failure disease.

In a first aspect, the heart failure disease is one of: heart failure with preserved ejection fraction (HFPEF); heart failure with ejection fraction ≧40%; diastolic heart failure; heart failure with elevated levels of TNF-α, IL-6, CRP, or TGF-β; hypertension with risk of developing HFPEF; atrial fibrillation with risk of developing HFPEF; diabetes with risk of developing HFPEF; COPD with risk of developing HFPEF; ischemic heart disease with risk of developing HFPEF; obesity with risk of developing HFPEF; chronic heart failure; compensated heart failure; and decompensated heart failure. In some embodiments, heart failure disease is heart failure with preserved ejection fraction.

FAFDs to be used in the treatment of HFPEF are molecular conjugates. A molecular conjugate comprises a fumarate and a fatty acid wherein the fatty acid is selected from the group consisting of omega-3 fatty acids, fatty acids that are metabolized in vivo to omega-3 fatty acids, and lipoic acid, and the conjugate is capable of hydrolysis to produce free fumarate and free fatty acid. In some embodiments, the fatty acid is selected from the group consisting of all-cis-7, 10, 13-hexadecatrienoic acid, α-linolenic acid, stearidonic acid, eicosatrienoic acid, eicosatetraenoic acid, eicosapentaenoic acid (EPA), docosapentaenoic acid, docosahexaenoic acid (DHA), tetracosapentaenoic acid, tetracosahexaenoic acid and lipoic acid. In other embodiments, the fatty acid is selected from eicosapentaenoic acid, docosahexaenoic acid and lipoic acid. In some embodiments, the hydrolysis is enzymatic.

The present disclosure provides methods of treating a heart failure disease, including IIFPEF, by administering a FAFD of Formula (I) or (II), and pharmaceutical compositions containing a FAFD of Formula (I) or (II).

In another aspect, the methods disclosed herein use FAFDs of the Formula I and Formula II:

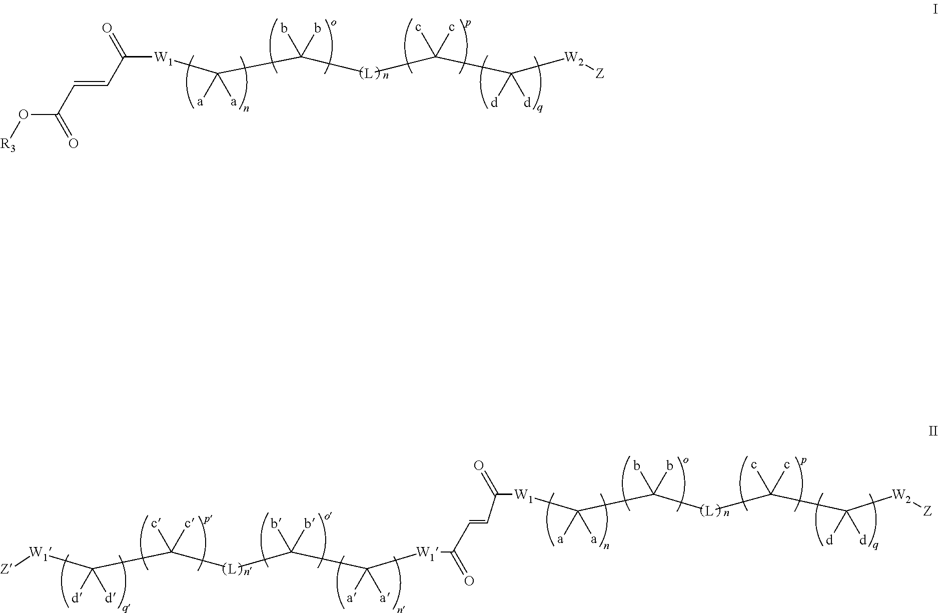

and pharmaceutically acceptable salts, hydrates, solvates, prodrugs, enantiomers, and stereoisomers thereof;

wherein

each W1, W2, W1′, and W2′ is independently null, O, S, NH, or NR, or W1 and W2, or W1′ and W2′ can be taken together to form an optionally substituted imidazolidine or piperazine group;

each a, b, c, d, a′, b′, c′, and d′ is independently —H, —D, —CH3, —OCH3, —OCH2CH3, —C(O)OR, —O—Z, or benzyl, or two of a, b, c, and d or any two of a′, b′, c′, and d′ can be taken together, along with the single carbon to which they are bound, to form a cycloalkyl or heterocycle;

each n, o, p, q, n′, o′, p′, and q′ is independently 0, 1, or 2;

each L and L′ is independently null, —O—, —C(O)—, —S—, —S(O)—, —S(O)2—, —S—S—, —(C1-C6 alkyl)—, —(C3-C6 cycloalkyl)-, a heterocycle, a heteroaryl,

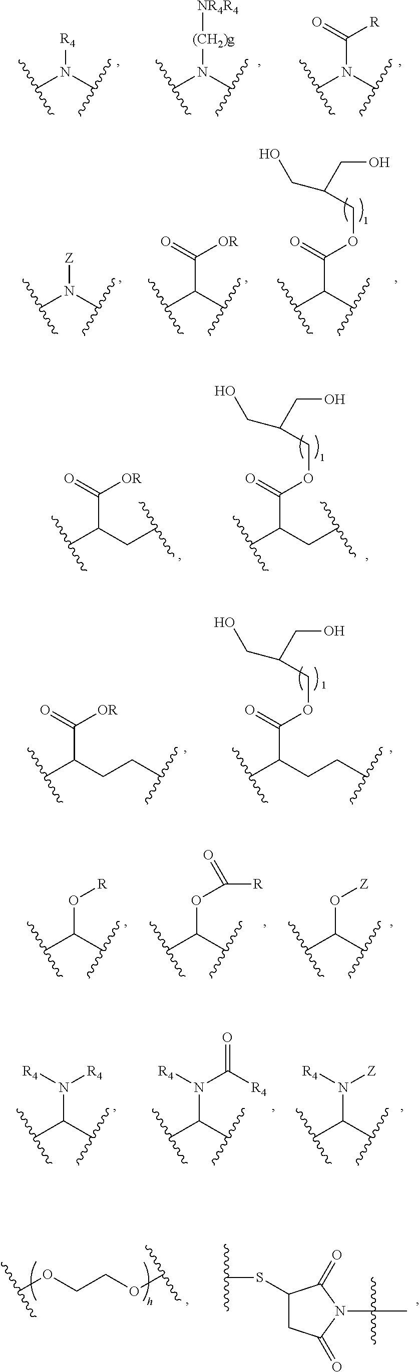

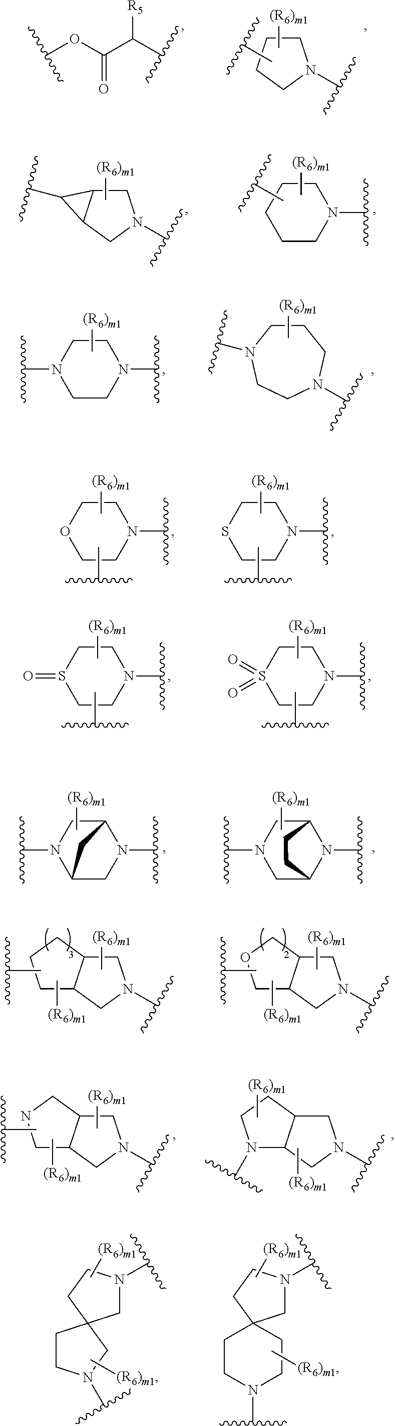

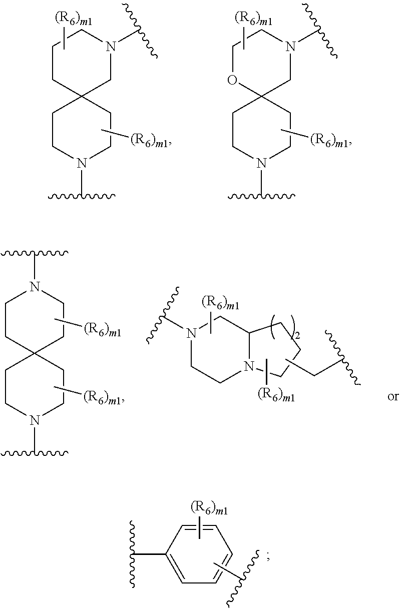

wherein the representation of L and L′ is not limited directionally left to right as is depicted, rather either the left side or the right side of L and L′ can be bound to the W1 or W1′ side of the compound of Formula I or Formula II, respectively;

each R6 is independently —H, —D, —C1-C4 alkyl, -halogen, cyano, oxo, thiooxo, —OH, —C(O)C1-C4 alkyl, —O-aryl, —O-benzyl, —OC(O)C1-C4 alkyl, —C1-C3 alkene, —C1-C3 alkyne, —C(O)C1-C4 alkyl, —NH2, —NH(C1-C3 alkyl), —N(C1-C3 alkyl)2, —NH(C(O)C1-C3 alkyl), —N(C(O)C1-C3 alkyl)2, —SH, —S(C1-C3 alkyl), —S(O)C1-C3 alkyl, —S(O)2C1-C3 alkyl;

each g is independently 2,3, or 4;

each h is independently 1,2, 3, or 4;

each m and m′ is independently 0, 1, 2, or 3; if m or m′ is more than 1, then L or L′ can be the same or different;

each m1 is independently 0, 1, 2, or 3;

k is 0, 1, 2, or 3;

z is 1, 2, or 3;

each R4 is independently H or optionally substituted C1-C6 alkyl, wherein a methylene unit of the C1-C6 alkyl can be optionally substituted for either O or NR, and in NR4R4, both R4 when taken together with the nitrogen to which they are attached can form a heterocyclic ring such as a pyrrolidine, piperidine, morpholine, piperazine or pyrrole;

each Z and Z′ is independently H,

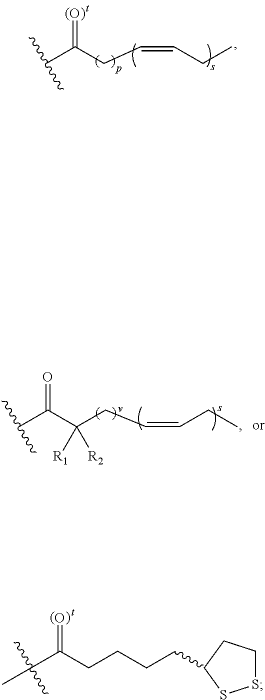

Provided that there is at least one of the following:

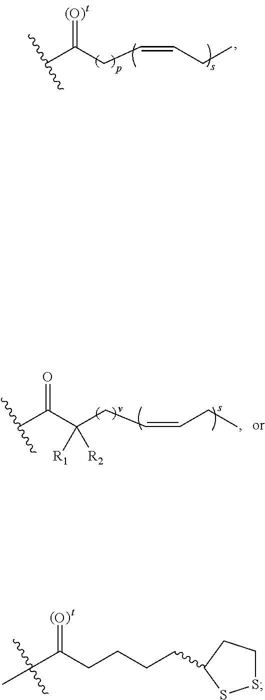

is in the compound;

each t is independently 0 or 1;

each r is independently 2, 3, or 7;

each s is independently 3,5, or 6;

each v is independently 1, 2, or 6;

each R1 and R2 is independently —H, —D, —C1-C4 alkyl, -halogen, —OH, —C(O)C1-C4 alkyl, —O-aryl, —O-benzyl, —OC(O)C1-C4 alkyl, —C1-C3 alkene, —C1-C3 alkyne, —C(O)C1-C4 alkyl, —NH2, —NH(C1-C3 alkyl), —N(C1-C3 alkyl)2, —NH(C(O)C1-C3 alkyl), —N(C(O)C1-C3 alkyl)2, —SH, —S(C1-C3 alkyl), —S(O)C1-C3 alkyl, —S(O)2C1-C3 alkyl;

each R3 is independently H, —C1-C6 alkyl or —C(CH2OH)2;

each R5 is independently e, H or straight or branched C1-C10 alkyl which can be optionally substituted with OH, NH2, C02R, CONH2, phenyl, C6H40H, imidazole or arginine;

each e is independently H or any one of the side chains of the naturally occurring amino acids;

each R is independently —H or straight or branched C1-C4 alkyl optionally substituted with OH or halogen;

provided that when each of m, n, o, p, and q, is 0, W1 and W2 is each null, and Z is

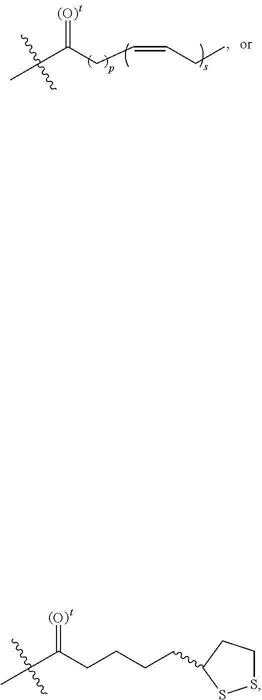

then t must be 0;

when each of m′, n′, o′, p′, and q is 0, W1′ and W2′ is each null, and Z′ is

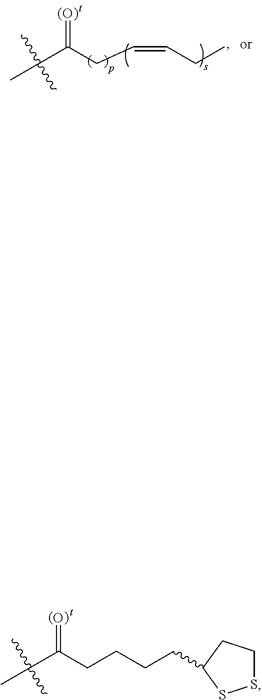

then t must be 0; and when each of m, n, o, p, and q is 0, and W1 and W2 is each null, or when each of m′, n′, o′, p′, and q′, is 0, W1′ and W2′ is each null, then Z or Z′ must not be

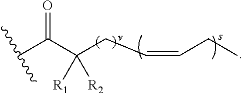

In another aspect, methods disclosed herein use FAFDs of Formula IA:

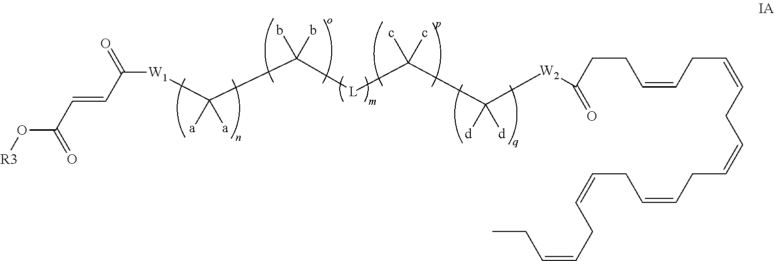

and pharmaceutically acceptable salts, hydrates, solvates, prodrugs, enantiomers, and stereoisomers thereof;

wherein each W1 and W2 is independently null, O, S, NH, or NR, or W1 and W2 can be taken together to form an optionally substituted imidazolidine or piperazine group;

each a, b, c, and d is independently —H, —D, —CH3, —OCH3, —OCH2CH3, —C(O)OR, or benzyl, or two of a, b, c, and d can be taken together, along with the single carbon to which they are bound, to form a cycloalkyl or heterocycle;

each n, o, p, and q is independently 0, 1, or 2;

each L is independently null, —O—, —O(O)—, —S—, —S(O)—, —S(O)2—, —S—S—, -(C1-C6 alkyl)-, -(C3-C6 cycloalkyl)-, a heterocycle, a heteroaryl,

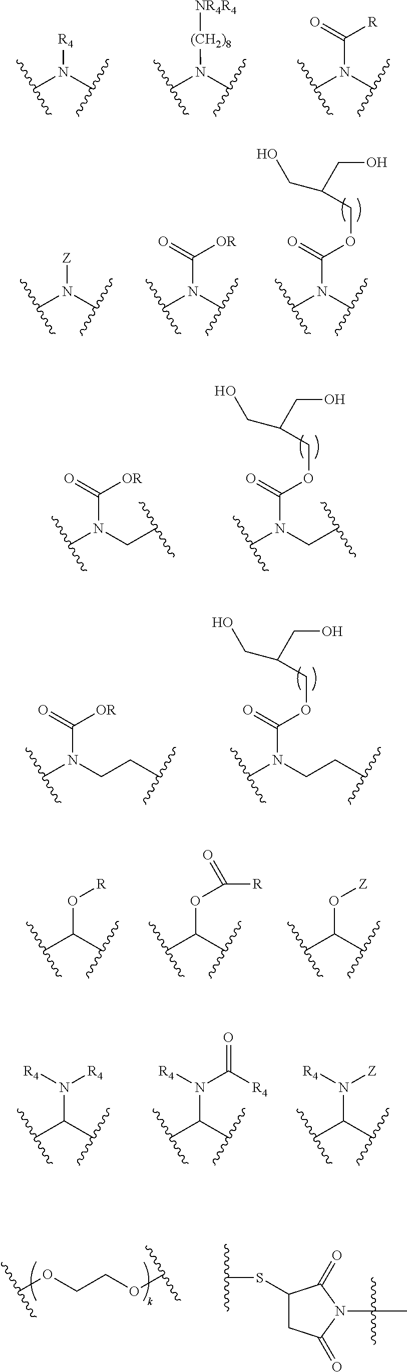

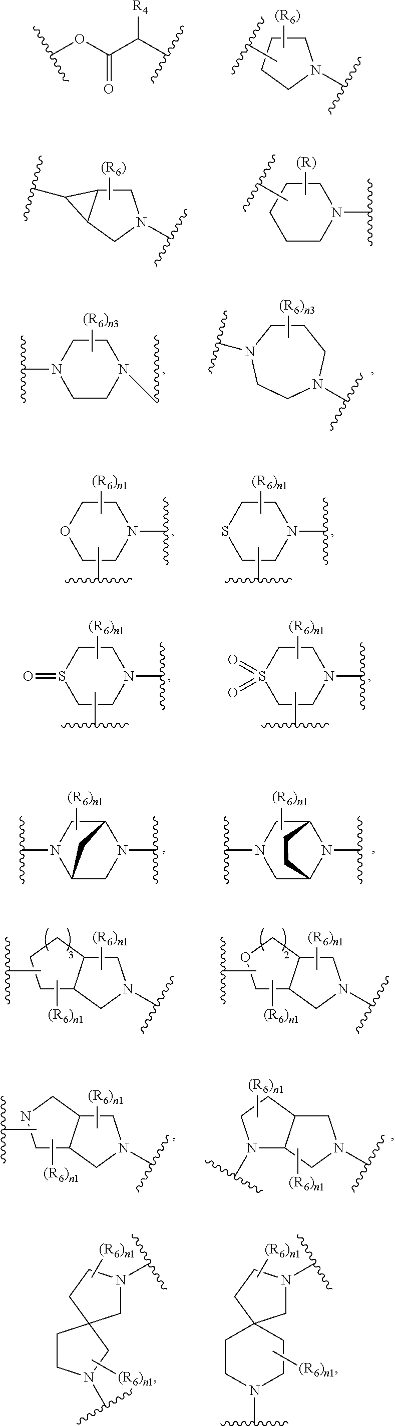

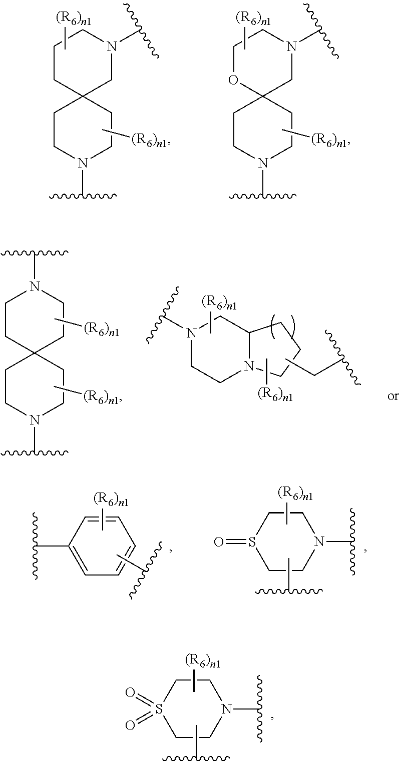

wherein the representation of L is not limited directionally left to right as is depicted, rather either the left side or the right side of L can be bound to the W1 side of the compound of Formula IA;

each R6 is independently —H, —D, —C1-C4 alkyl, -halogen, cyano, oxo, thiooxo, —OH, —C(O)C1-C4 alkyl, —O-aryl, —O-benzyl, —OC(O)C1-C4 alkyl, —C1-C3 alkene, —C1-C3 alkyne, —C(O)C1-C4 alkyl, —NH2, —NH(C1-C3 alkyl), —N(C1-C3 alkyl)2, —NH(C(O)C1-C3 alkyl), —N(C(O)C1-C3 alkyl)2, —SH, —S(C1-C3 alkyl), —S(O)C1-C3 alkyl, —S(O)2C1-C3 alkyl;

each g is independently 2, 3, or 4;

each h is independently 1, 2, 3, or 4; each m is independently 0, 1, 2, or 3; if m is more than 1, then L can be the same or different;

each m1 is independently 0, 1, 2, or 3;

k is 0, 1, 2, or 3;

z is 1, 2, or 3;

each R4 is independently H or optionally substituted C1-C6 alkyl, wherein a methylene unit of the C1-C6 alkyl can be optionally substituted for either O or NR, and in NR4R4, both R4 when taken together with the nitrogen to which they are attached can form a heterocyclic ring such as a pyrrolidine, piperidine, morpholine, piperazine or pyrrole;

each R3 is independently H, —C1-C6 alkyl or —C(CH2OH)2;

each R5 is independently e, H or straight or branched C1-C10 alkyl which can be optionally substituted with OH, NH2, CO2R, CONH2, phenyl, C6H4OH, imidazole or arginine;

each e is independently H or any one of the side chains of the naturally occurring amino acids;

each R is independently —H, or straight or branched C1-C4 alkyl optionally substituted with OH, or halogen.

In another aspect, methods disclosed herein use FAFDs of Formula IB:

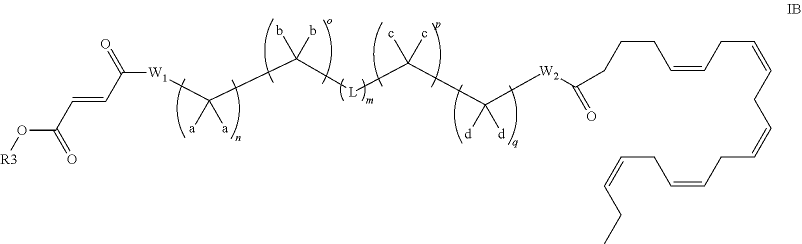

and pharmaceutically acceptable salts, hydrates, solvates, prodrugs, enantiomers, and stereoisomers thereof;

wherein

each W1 and W2 is independently null, O, S, NH, or NR, or W1 and W2 can be taken together can form an optionally substituted imidazolidine or piperazine group;

each a, b, c, and d is independently —H, —D, —CH3, —OCH3, —OCH2CH3, —C(O)OR, or benzyl, or two of a, b, c, and d can be taken together, along with the single carbon to which they are bound, to form a cycloalkyl or heterocycle;

each n, o, p, and q is independently 0, 1, or 2;

each L is independently null, —O—, —C(O)—, —S—, —S(O)—, —S(O)2—, —S—S—, —(C1-C6 alkyl)-, -(C3-C6 cycloalkyl)-, a heterocycle, a heteroaryl,

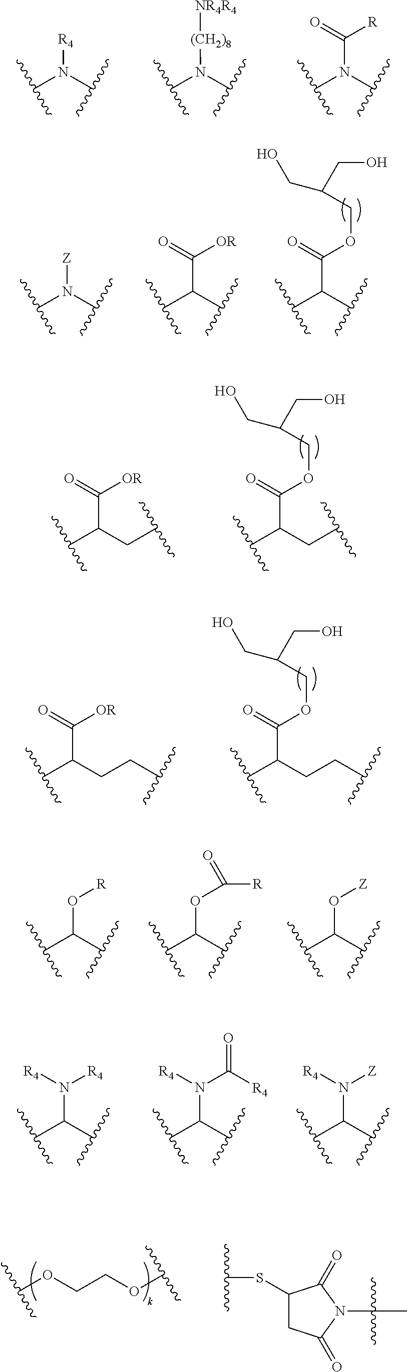

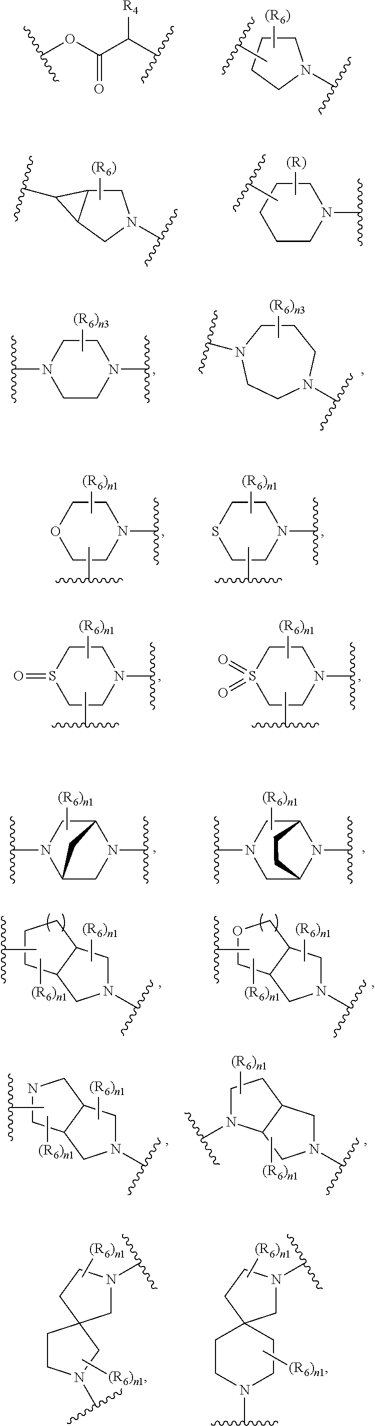

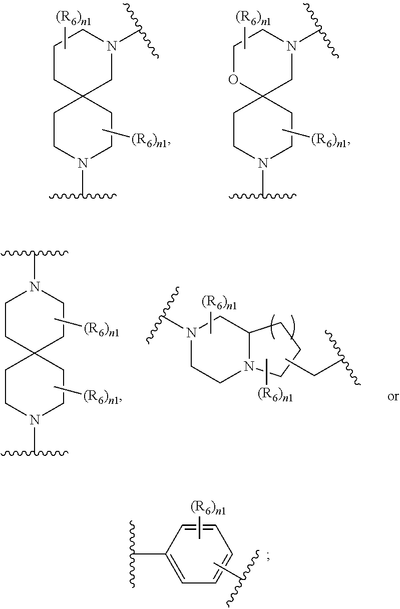

wherein the representation of L is not limited directionally left to right as is depicted, rather either the left side or the right side of L can be bound to the W1 side of the compound of Formula IB;

each R6 is independently —H, —D, —C1-C4 alkyl, -halogen, cyano, oxo, thiooxo, —OH, —C(O)C1-C4 alkyl, —O-aryl, —O-benzyl, —O C(O)C1-C4 alkyl, —C1-C3 alkene, - C1-C3 alkyne, —C(O)C1-C4 alkyl, —NH2, —NH(C1-C3 alkyl), —N(C1-C3 alkyl)2, —NH(C(O)C1-C3 alkyl), —N(C(O)C1-C3 alkyl)2, —SH, —S(C1-C3 alkyl), —S(O)C1-C3 alkyl, —S(O)2C1-C3 alkyl;

each g is independently 2,3, or 4;

each h is independently 1, 2, 3, or 4;

each m is independently 0, 1, 2, or 3; if m is more than 1, then L can be the same or different;

each ml is independently 0, 1, 2 or 3;

k is 0, 1,2, or 3;

z is 1, 2, or 3;

each R4 is independently H or optionally substituted C1-C6 alkyl, wherein a methylene unit of the C1-C6 alkyl can be optionally substituted for either O or NR, and in NR4R4, both R4 when taken together with the nitrogen to which they are attached can form a heterocyclic ring such as a pyrrolidine, piperidine, morpholine, piperazine or pyrrole;

each R3 is independently H, —C1-C6 alkyl or —C(CH2OH)2;

each R5 is independently e, H or straight or branched C1-C10 alkyl which can be optionally substituted with OH, NH2, CO2R, CONH2, phenyl, C6H4OH, imidazole or arginine;

each e is independently H or any one of the side chains of the naturally occurring amino acids;

each R is independently —H, or straight or branched C1-C4 alkyl optionally substituted with OH, or halogen.

In another aspect, the methods disclosed herein use FAFDs of Formula IC:

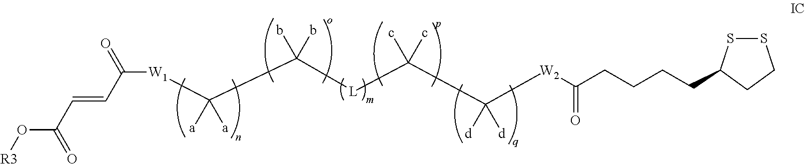

and pharmaceutically acceptable salts, hydrates, solvates, prodrugs, enantiomers, and stereoisomers thereof;

wherein

each W1 and W2 is independently null, O, S, NH, or NR, or W1 and W2 can be taken together can form an optionally substituted imidazolidine or piperazine group;

each a, b, c, and d is independently —H, —D, —CH3, —OCH3, —OCH2CH3, —C(O)OR, or benzyl, or two of a, b, c, and d can be taken together, along with the single carbon to which they are bound, to form a cycloalkyl or heterocycle;

each n, o, p, and q is independently 0,1, or 2;

each L is independently null, —O—, —C(O)—, —S—, —S(O)—, —S(O)2—, —S—S—, -(C1-C6 alkyl)-, -(C3-C6 cycloalkyl)-, a heterocycle, a heteroaryl,

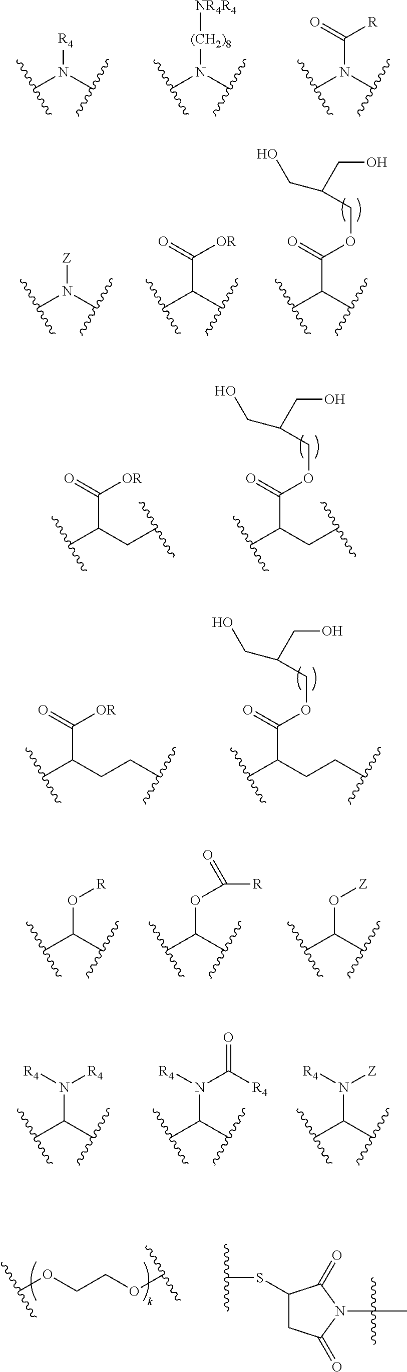

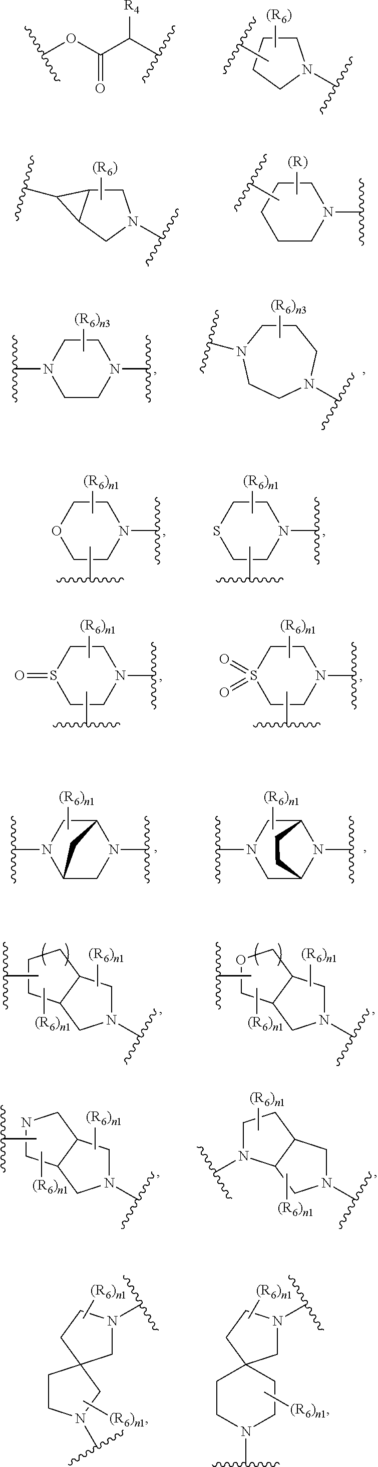

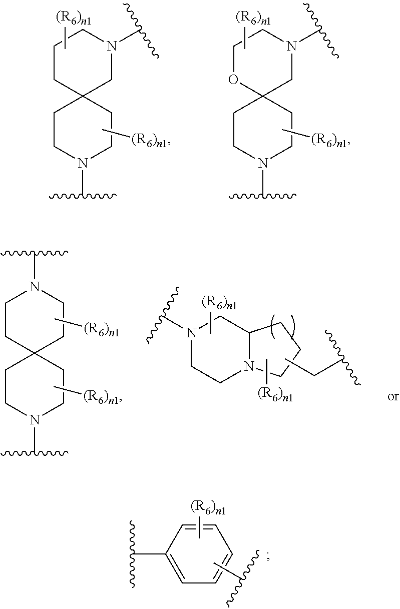

wherein the representation of L is not limited directionally left to right as is depicted, rather either the left side or the right side of L can be bound to the W1 side of the compound of Formula IC;

each R6 is independently —H, —D, —C1-C4 alkyl, -halogen, cyano, oxo, thiooxo, —OH, —C(O)C1-C4 alkyl, —O-aryl, —O-benzyl, —OC(O)C1-C4 alkyl, —C1-C3 alkene, - C1-C3 alkyne, —C(O)C1-C4 alkyl, —NH2, —NH(C1-C3 alkyl), —N(C1-C3 alkyl)2, —NH(C(O)C1-C3 alkyl), —N(C(O)C1-C3 alkyl)2, —SH, —S(C1-C3 alkyl), —S(O)C1-C3 alkyl, —S(O)2C1-C3 alkyl;

each g is independently 2, 3, or 4;

each h is independently 1, 2, 3, or 4;

each m is independently 0, 1, 2, or 3; if m is more than 1, then L can be the same or different;

each m1 is independently 0, 1, 2, or 3;

k is 0, 1,2, or 3;

z is 1,2, or 3;

each R4 is independently H or optionally substituted C1-C6 alkyl, wherein a methylene unit of the C1-C6 alkyl can be optionally substituted for either O or NR, and in NR4R4, both R4 when taken together with the nitrogen to which they are attached can form a heterocyclic ring such as a pyrrolidine, piperidine, morpholine, piperazine or pyrrole;

each R3 is independently H, —C1-C6 alkyl or —C(CH2OH)2;

each R5 is independently e, H or straight or branched C1-C10 alkyl which can be optionally substituted with OH, NH2, CO2R, CONH2, phenyl, C6H4OH, imidazole or arginine;

each e is independently H or any one of the side chains of the naturally occurring amino acids;

each R is independently —H, or straight or branched C1-C4 alkyl optionally substituted with OH or halogen.

In compounds of Formula I, IA, IB, IC, and II, any one or more of H may be substituted with a deuterium. It is also understood that in compounds of Formula I, IA, IB, IC, and II, that a methyl substituent can be substituted with a C1-C6 alkyl.

In one embodiment of the present disclosure, methods disclosed herein use FAFD of formula (E)-methyl 4-(2-(4Z,7Z,10Z,13Z,16Z,19Z)-docosa-4,7,10,13,16,19-hexaenamidoethylamino)-4-oxobut-2-enoate (I-1) or a pharmaceutically acceptable salt thereof.

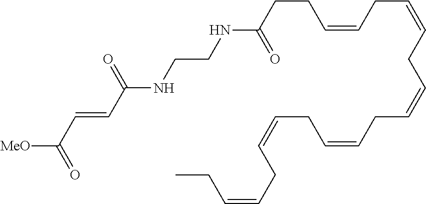 [SMILES: CC/C=C\C/C=C\C/C=C\C/C=C\C/C=C\C/C=C\CCC(=O)NCCNC(=O)/C=C/C(=O)OC]

The present disclosure also provides pharmaceutical formulations comprising at least one FAFDas described in U.S. Pat. No. 8,969,354, the disclosure of which is herein incorporated by reference in its entirety, and one or more pharmaceutically acceptable carriers for the treatment of heart failure disease. In some embodiments, the heart failure disease is heart failure with preserved ejection fraction (HFPEF).

In another embodiment, a pharmaceutical composition is administered to the subject, wherein said pharmaceutical composition comprises about 20 mg to about 5000 mg of (E)-methyl 4-(2-(4Z,7Z, 10Z, 13Z, 16Z, 19Z)-docosa-4,7,10,13,16,19-hexaenamidoethylamino)-4-oxobut-2-enoate (I-1) or a pharmaceutically acceptable salt thereof.

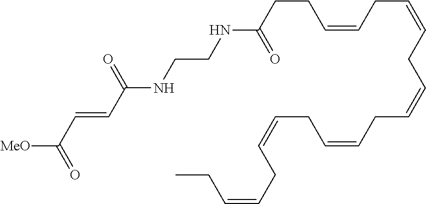 [SMILES: CC/C=C\C/C=C\C/C=C\C/C=C\C/C=C\C/C=C\CCC(=O)NCCNC(=O)/C=C/C(=O)OC]

In some embodiments, a pharmaceutical composition is administered to the subject, wherein said pharmaceutical composition comprises a therapeutically effective amount of (E)-methyl 4-(2-(4Z,7Z,10Z,13Z,16Z,19Z)-docosa-4,7,10,13,16,19-hexaenamidoethylamino)-4-oxobut-2-enoate (I-1) or a pharmaceutically acceptable salt thereof that is shown to provide MMF plasma exposure comparable to dimethyl fumarate (DMF) 120 mg to 720 mg per day.

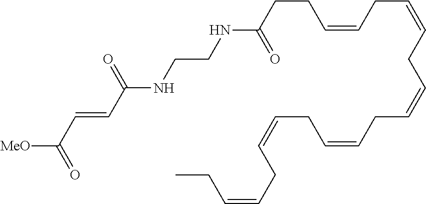 [SMILES: CC/C=C\C/C=C\C/C=C\C/C=C\C/C=C\C/C=C\CCC(=O)NCCNC(=O)/C=C/C(=O)OC]

In one embodiment, (E)-methyl 4-(2-(4Z,7Z,10Z,13Z,16Z,19Z)-docosa-4,7,10,13,16,19-hexaenamidoethylamino)-4-oxobut-2-enoate (I-1) or a pharmaceutically acceptable salt thereof is administered in combination with one or more second agents useful for treating heart failure .In various embodiment, the second agent is selected from the group consisting of: a diuretic, an ace-inhibitor, a beta-blocker, an angiotensin receptor blocker, isosorbide dinitrate, hydralazine, an angiotensin receptor-neprilysin inhibitor, an aldosterone antagonist, a PDE5 inhibitor, a statin, a neprilysin inhibitor, an aldosterone inhibitor, and an antitumor necrosis factor-alpha therapy. In one embodiment, the second agent is a statin.

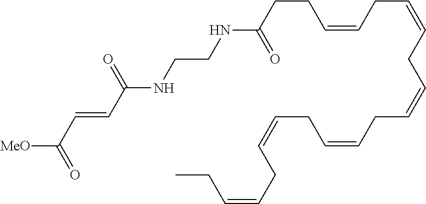 [SMILES: CC/C=C\C/C=C\C/C=C\C/C=C\C/C=C\C/C=C\CCC(=O)NCCNC(=O)/C=C/C(=O)OC]

Another aspect of the present disclosure provides for a pharmaceutical composition comprising (a) (E)-methyl 4-(2-(4Z,7Z,10Z,13Z,16Z,19Z)-docosa-4,7,10,13,16,19-hexaenamidoethylamino)-4-oxobut-2-enoate (I-1) or a pharmaceutically acceptable salt and (b) a statin and one or more pharmaceutically acceptable excipients.

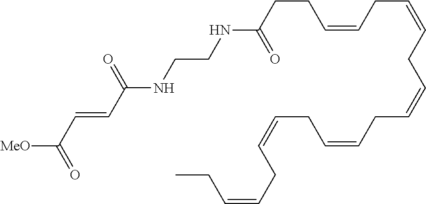 [SMILES: CC/C=C\C/C=C\C/C=C\C/C=C\C/C=C\C/C=C\CCC(=O)NCCNC(=O)/C=C/C(=O)OC]

In some embodiments, the pharmaceutical composition comprises (E)-methyl 4-(2-(4Z,7Z,10Z,13Z,16Z,19Z)-docosa-4,7,10,13,16,19-hexaenamidoethylamino)-4-oxobut- 2-enoate (I-1) or a pharmaceutically acceptable salt thereof at a dose range of about 20 mg to about 5000 mg of the FAFD per day. Compositions for in vivo or in vitro use can contain about 20, 50, 75, 100, 150, 250, 500, 750, 1000, 1250, 2500, 3500, or 5000 mg of the FAFD. In one embodiment, the composition is in the form of a tablet that can be scored. Effective plasma levels of the FAFD can range from about 0.002 mg to about 100 mg per kg of body weight per day, for example as described in U.S. patent application Ser. No. 15/075,829, the disclosure of which is incorporated by reference in its entirety.

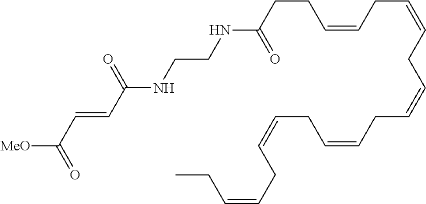 [SMILES: CC/C=C\C/C=C\C/C=C\C/C=C\C/C=C\C/C=C\CCC(=O)NCCNC(=O)/C=C/C(=O)OC]

Another aspect of the disclosure provides a method of treating a heart failure disease in a subject in need thereof, the method comprising: administering to the subject a therapeutically effective amount of (a) (E)-methyl 4-(2-(4Z,7Z,10Z,13Z,16Z,19Z)-docosa-4,7,10,13,16,19-hexaenamidoethylamino)-4-oxobut-2-enoate (I-1) and either separately or together with (b) a statin.

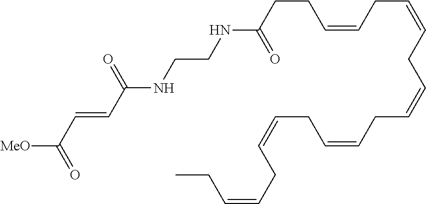 [SMILES: CC/C=C\C/C=C\C/C=C\C/C=C\C/C=C\C/C=C\CCC(=O)NCCNC(=O)/C=C/C(=O)OC]

The details of the invention are set forth in the accompanying description below.

Although methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this invention belongs. All patents and publications cited in this specification are incorporated herein by reference in their entireties.

### DETAILED DESCRIPTION

The FAFDs possess the ability to treat or prevent heart failure diseases, including heart failure with preserved ejection fraction (HFPEF).

The FAFDs have been designed to bring together fumaric acid and ester analogs thereof and fatty acids into a single molecular conjugate. The activity of the FAFDs is substantially greater than the sum of the components suggesting that the activity induced by the FAFDs is synergistic.

### Definitions

The following definitions are used in connection with the FAFDs.

The term “fatty acid fumarate derivatives” of “FAFD” includes any and all possible isomers, stereoisomers, enantiomers, diastereomers, tautomers, pharmaceutically acceptable salts, hydrates, solvates, and prodrugs of the FAFDs described herein.

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

Unless otherwise specifically defined, the term “aryl” refers to cyclic, aromatic hydrocarbon groups that have 1 to 2 aromatic rings, including monocyclic or bicyclic groups such as phenyl, biphenyl or naphthyl. Where containing two aromatic rings (bicyclic, etc.), the aromatic rings of the aryl group may be joined at a single point (e.g., biphenyl) or fused (e.g., naphthyl). The aryl group may be optionally substituted by one or more substituents, e.g., 1 to 5 substituents, at any point of attachment. The substituents can themselves be optionally substituted.

“C₁-C₃ alkyl” refers to a straight or branched chain saturated hydrocarbon containing 1-3 carbon atoms. Examples of a C₁-C₃ alkyl group include, but are not limited to, methyl, ethyl, propyl and isopropyl.

“C₁-C₄ alkyl” refers to a straight or branched chain saturated hydrocarbon containing 1-4 carbon atoms. Examples of a C₁-C₄ alkyl group include, but are not limited to, methyl, ethyl, propyl, butyl, isopropyl, isobutyl, sec-butyl and tert-butyl.

“C₁-C₅ alkyl” refers to a straight or branched chain saturated hydrocarbon containing 1-5 carbon atoms. Examples of a C₁-C₅ alkyl group include, but are not limited to, methyl, ethyl, propyl, butyl, pentyl, isopropyl, isobutyl, sec-butyl and tert-butyl, isopentyl and neopentyl.

“C₁-C₆ alkyl” refers to a straight or branched chain saturated hydrocarbon containing 1-6 carbon atoms. Examples of a C₁-C₆ alkyl group include, but are not limited to, methyl, ethyl, propyl, butyl, pentyl, hexyl, isopropyl, isobutyl, sec-butyl, tert-butyl, isopentyl, and neopentyl.

The term “cycloalkyl” refers to a cyclic hydrocarbon containing 3-6 carbon atoms. Examples of a cycloalkyl group include, but are not limited to, cyclopropyl, cyclobutyl, cyclopentyl and cyclohexyl. It is understood that any of the substitutable hydrogens on a cycloalkyl can be substituted with halogen, C₁-C₃ alkyl, hydroxyl, alkoxy and cyano groups.

The term “heterocycle” as used herein refers to a monocyclic or bicyclic hydrocarbon containing 3-12 carbon atoms wherein at least one of the carbon atoms is substituted with a O, N, or S. Examples of a heterocycle include, but are not limited to, aziridine, oxirane, thiirane, azetidine, oxetane, thietane, pyrrolidine, tetrahydrofurane, tetrahydrothiophene, piperidine, tetrahydropyran, thiane, imidazolidine, oxazolidine, thiazolidine, dioxolane, dithiolane, piperazine, oxazine, dithiane, dioxane, diazabicycloheptane and diazabicyclooctane.

The term “heteroaryl” as used herein refers to a monocyclic or bicyclic ring structure having 5 to 12 ring atoms wherein one or more of the ring atoms is a heteroatom, e.g. N, O or S and wherein one or more rings of the bicyclic ring structure is aromatic. Some examples of heteroaryl are pyridyl, furyl, pyrrolyl, thienyl, thiazolyl, oxazolyl, imidazolyl, indolyl, tetrazolyl, benzofuryl, xanthenes and dihydroindole. It is understood that any of the substitutable hydrogens on a heteroaryl can be substituted with halogen, C₁-C₃ alkyl, hydroxyl, alkoxy and cyano groups.

The term “any one of the side chains of the naturally occurring amino acids” as used herein means a side chain of any one of the following amino acids: Isoleucine, Alanine, Leucine, Asparagine, Lysine, Aspartate, Methionine, Cysteine, Phenylalanine, Glutamate, Threonine, Glutamine, Tryptophan, Glycine, Valine, Proline, Arginine, Serine, Histidine, and Tyrosine.

The term “fatty acid” as used herein means an omega-3 fatty acid, fatty acids that are metabolized in vivo to omega-3 fatty acids, and lipoic acid. Non-limiting examples of fatty acids are all-cis-7,10,13-hexadecatrienoic acid, α-linolenic acid (ALA or all-cis-9,12,15-octadecatrienoic acid), stearidonic acid (STD or all-cis-6,9,12,15-octadecatetraenoic acid), eicosatrienoic acid (ETE or all-cis-11,14,17-eicosatrienoic acid), eicosatetraenoic acid (ETA or all-cis-8,11,14,17-eicosatetraenoic acid), eicosapentaenoic acid (EPA or all-cis-5,8,11,14,17-eicosapentaenoic acid), docosapentaenoic acid (DPA, clupanodonic acid or all-cis-7,10,13,16,19-docosapentaenoic acid), docosahexaenoic acid (DHA or all-cis-4,7,10,13,16,19-docosahexaenoic acid), tetracosapentaenoic acid (all-cis-9,12,15,18,21 -docosahexaenoic acid), tetracosahexaenoic acid (nisinic acid or all-cis-6,9,12,15,18,21-tetracosenoic acid) and stereoisomers of lipoic acid.

A “subject” is a mammal, e.g., a human, mouse, rat, guinea pig, dog, cat, horse, cow, pig, or non-human primate, such as a monkey, chimpanzee, baboon or rhesus.

The invention also includes pharmaceutical compositions comprising an effective amount of a FAFD and a pharmaceutically acceptable carrier. The invention includes a FAFD when provided as a pharmaceutically acceptable prodrug, hydrate, salt, such as a pharmaceutically acceptable salt, enantiomers, stereoisomers, or mixtures thereof.

Representative “pharmaceutically acceptable salts” include, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, subsalicylate, suramate, tannate, tartrate, teoclate, tosylate, triethiodide, and valerate salts.

The term “heart failure disease” may be heart failure with preserved ejection fraction (HFPEF); heart failure with ejection fraction ≧40%; diastolic heart failure; heart failure with elevated levels of TNF-α, IL-6, CRP, or TGF-β; hypertension with a risk of developing HFPEF; atrial fibrillation with a risk of developing HFPEF; diabetes with a risk of developing HFPEF; COPD with a risk of developing HFPEF; ischemic heart disease with a risk of developing HFPEF; obesity with a risk of developing HFPEF; chronic heart failure; compensated heart failure; decompensated heart failure; or other conditions known to have a high risk of developing HFPEF. In particular, heart failure disease is heart failure with preserved ejection fraction (HFPEF).

An “effective amount” when used in connection with a FAFDis an amount effective for treating or preventing a metabolic disorder.

The term “carrier,” as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body.

The term “treating,” with regard to a subject, refers to improving at least one symptom of the subject's disorder. Treating can be curing, improving, or at least partially ameliorating the disorder.

The term “disorder” is used in this disclosure to mean, and is used interchangeably with, the terms disease, condition, or illness, unless otherwise indicated.

The term “administer,” “administering,” or “administration” as used in this disclosure refers to either directly administering a compound or pharmaceutically acceptable salt of the compound or a composition to a subject, or administering a prodrug derivative or analog of the compound or pharmaceutically acceptable salt of the compound or composition to the subject, which can form an equivalent amount of active compound within the subject's body.

The term “prodrug,” as used in this disclosure, means a compound, which is convertible in vivo by metabolic means (e.g., by hydrolysis) to a FAFD.

### Compounds

Certain embodiments of methods disclosed herein use a FAFD according to Formulas I, IA, IB, IC, and II, as set forth below.

Certain embodiments of a method disclosed herein use a compound of Formula I and Formula II:

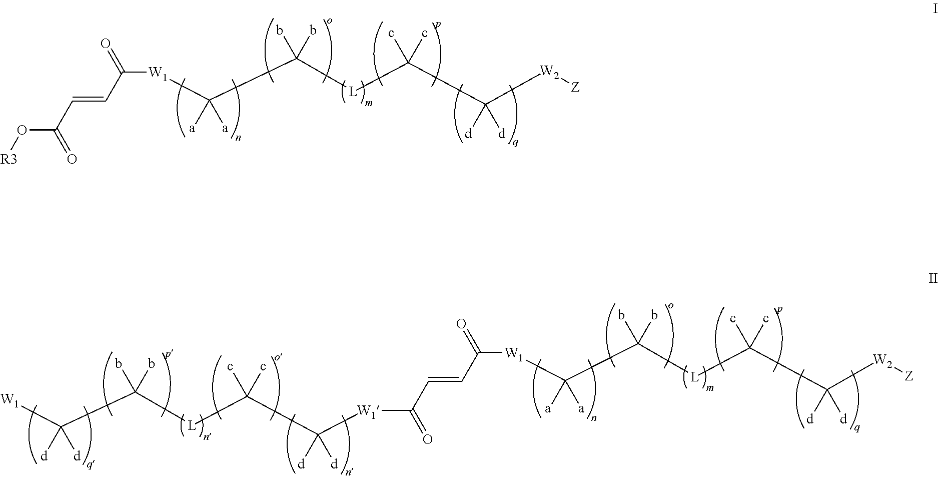

and pharmaceutically acceptable salts, hydrates, solvates, prodrugs, enantiomers, and stereoisomers thereof;

wherein

W1, W2, a, b, c, d, m, ml, n, o, p, q, L, Z, r, s, t, v, R, R1, R2, R3, R4, R5, R6, W1′, W2′, a′, c′, b′, d′, n′, o′, p′, q′, ml′, L′, and Z′ is as defined above for Formula I and Formula II, provided that there is at least one of the following in the compound.

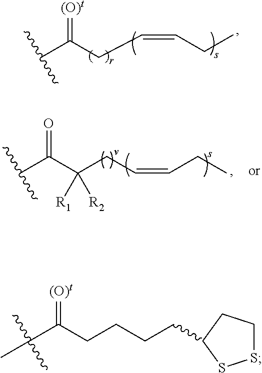

In some embodiments, one Z is

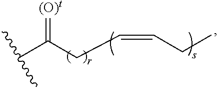

and r is 2.

In some embodiments, one Z is

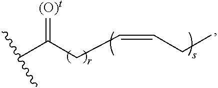

and r is 3.

In some embodiments, one Z is

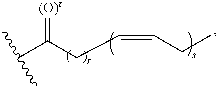

Tn other embodiments, one Z is

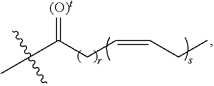

and s is 3.

In some embodiments, one Z is

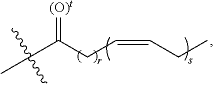

and s is 5.

In some embodiments, one Z is

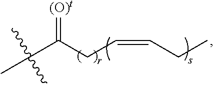

and s is 6.

In some embodiments, one Z is

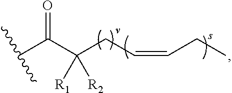

and v is 1.

In other embodiments, one Z is

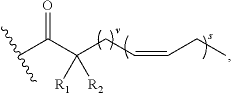

and v is 2.

In some embodiments, one Z is

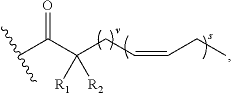

and v is 6.

In some embodiments, one Z is

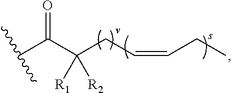

and s is 3.

In some embodiments, one Z is

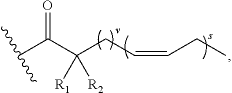

and sis 5.

In other embodiments, one Z is

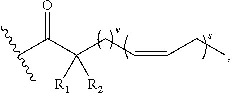

and sis 6.

In other embodiments, Z is

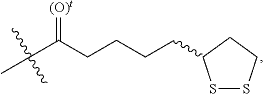

and t is 1.

In some embodiments, Z is

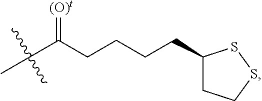

and t is 1.

In another aspect of methods disclosed herein use an compounds of Formula IA:

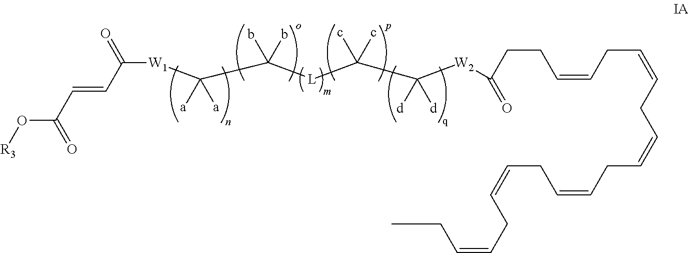

and pharmaceutically acceptable salts, hydrates, solvates, prodrugs, enantiomers, and stereoisomers thereof;

wherein

W1, W2, a, b, c, d, m, ml, n, o, p, q, L, R, R1, R2, R3, R4, R5, R6 are as defined above for Formula IA.

Certain embodiments of methods disclosed herein use a compound of Formula IB:

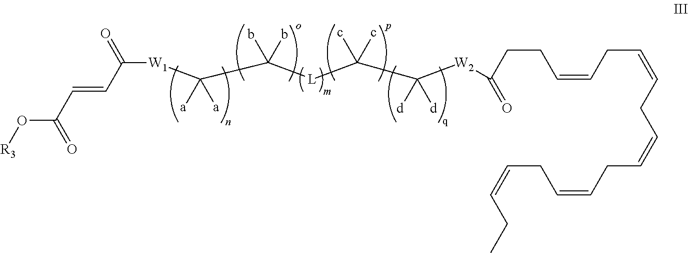

and pharmaceutically acceptable salts, hydrates, solvates, prodrugs, enantiomers, and stereoisomers thereof;

wherein

W1, W2, a, b, c, d, m, ml, n, o, p, q, L, R, R1, R2, R3, R4, R5, R6 are as defined above for Formula IB,

Certain embodiments of methods disclosed herein use a compound of Formula IC:

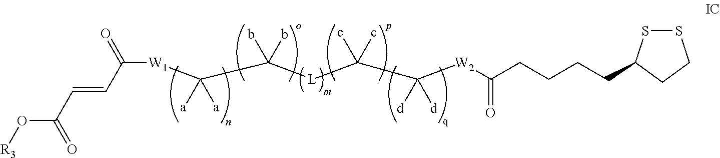

and pharmaceutically acceptable salts, hydrates, solvates, prodrugs, enantiomers, and stereoisomers thereof;

wherein

W1, W2, a, b, c, d, m, ml, n, o, p, q, L, R, R1, R2, R3, R4, R5, and R6 are as defined above for Formula IC.

The following embodiments are illustrative of a method of using compounds of Formulas I, IA, IB, IC, and II.

In some embodiments of a method of using compounds of Formula I, IA, IB, IC, and II, R3 is CH3.

In some embodiments of a method of using compounds of Formula I, IA, IB, IC, and II, R3 is —CH2CH3.

In some embodiments of a method of using compounds of Formula I, IA, IB, IC, and II, R3 is H.

In some embodiments of a method of using compounds of Formula I, IA, IB, IC, and II, W1 is NH.

In some embodiments of a method of using compounds of Formula I, IA, IB, IC, and II, W2 is NH.

In some embodiments of a method of using compounds of Formula I, IA, IB, IC, and II, Wt is O.

In some embodiments of a method of using compounds of Formula I, IA, IB, IC, and II, W2 is O.

In some embodiments of a method of using compounds of Formula I, IA, IB, IC, and II, W1 is null.

In some embodiments of a method of using compounds of Formula I, IA, IB, IC, and II, W2 is null.

In some embodiments of a method of using compounds of Formula I, IA, IB, IC, and II, W1 and W2 are each NH.

In some embodiments of a method of using compounds of Formula I, IA, IB, IC, and II, W1 and W2 are each null.

In some embodiments of a method of using compounds of Formula I, IA, IB, IC, and II, W1 is O and W2 are NH.

In some embodiments of a method of using compounds of Formula I, IA, IB, IC, and II, W1 and W2 is NR, and R are CH3.

In some embodiments of a method of using compounds of Formula I, IA, IB, IC, and II, m is 0.

In other embodiments of a method of using compounds of Formula I, IA, IB, IC, and II, m is 1.

In other embodiments of a method of using compounds of Formula I, IA, IB, IC, and II, m is 2.

In some embodiments of a method of using compounds of Formula I, IA, IB, IC, and II, L is —S— or —S—S—.

some embodiments of a method of using compounds of Formula I, IA, IB, IC, and II, L is —O-.

In some embodiments of a method of using compounds of Formula I, IA, IB, IC, and II, L is —C(O)-.

In some embodiments of a method of using compounds of Formula I, IA, IB, IC, and II, L is heteroaryl.

In some embodiments of a method of using compounds of Formula I, IA, IB, IC, and II, L is heterocycle.

In some embodiments of a method of using compounds of Formula I, IA, IB, IC, and II, L is

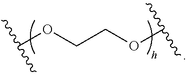

In some embodiments of a method of using compounds of Formula I, IA, IB, IC, and II, L is

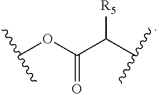

In some embodiments of a method of using compounds of Formula I, IA, IB, IC, and II, L is

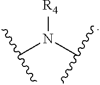

In some embodiments of a method of using compounds of Formula I, IA, IB, IC, and II, L is

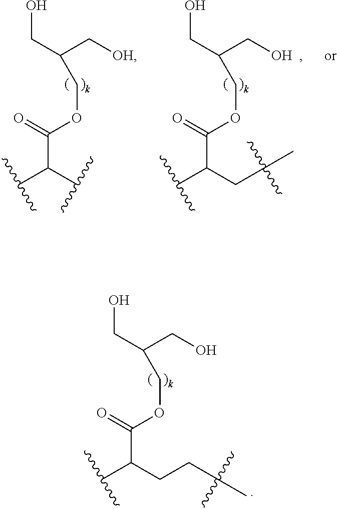

In some embodiments of a method of using compounds of Formula I, IA, IB, IC, and II, L is

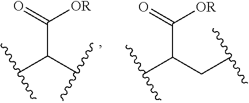

In some embodiments of a method of using compounds of Formula I, IA, IB, IC, and II, L is

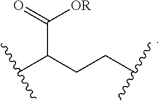

In some embodiments of a method of using compounds of Formula I, IA, IB, IC, and II, L is

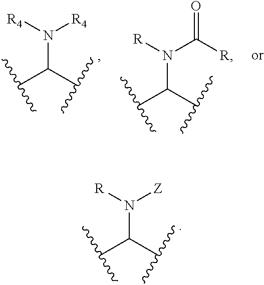

In some embodiments of a method of using compounds of Formula I, IA, IB, IC, and II, L is

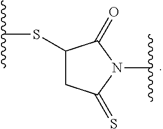 [SMILES: *SC1CC(=S)N(*)C1=O]

In some embodiments of a method of using compounds of Formula I, IA, IB, IC, and II, L is

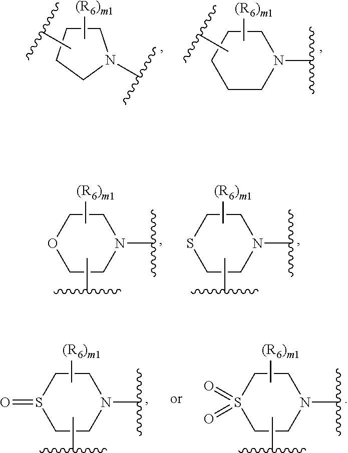

In some embodiments of a method of using compounds of Formula I, IA, IB, IC, and II, L is

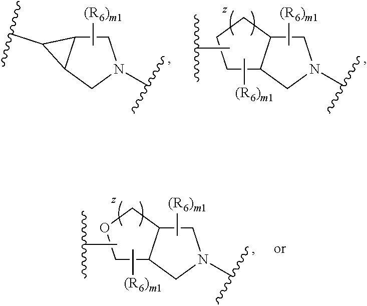

In some embodiments of a method of using compounds of Formula I, IA, IB, IC, and II, L is

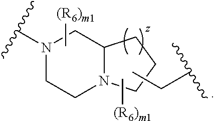

In some embodiments of a method of using compounds of Formula I, IA, IB, IC, and II, L is

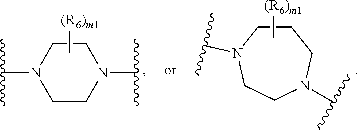

In some embodiments of a method of using compounds of Formula I, IA, IB, IC, and II, L is

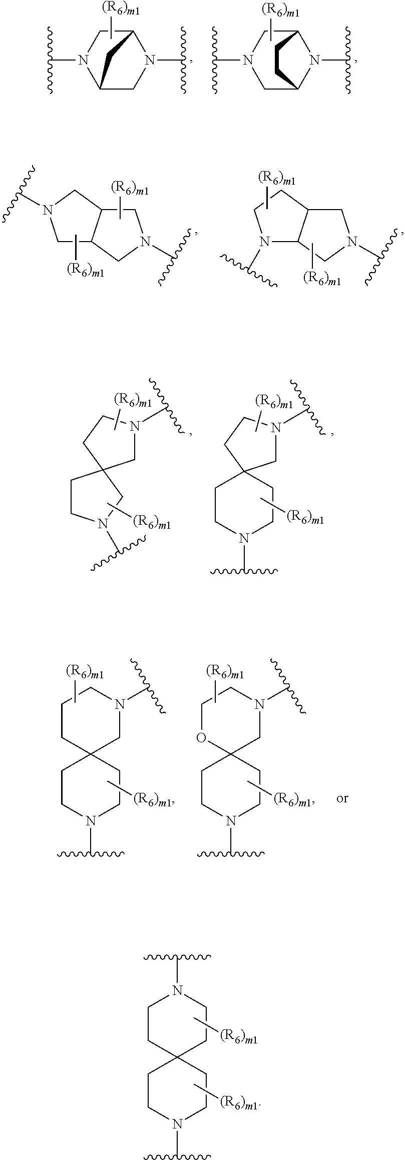

In other embodiments of a method of using compounds of Formula I, IA, IB, IC, and II, one of n, o, p, and q is 1.

In some embodiments of a method of using compounds of Formula I, IA, IB, IC, and II, two of n, o, p, and q are each 1.

In other embodiments of a method of using compounds of Formula I, IA, IB, IC, and II, three of n, o, p, and q are each 1.

In some embodiments of a method of using compounds of Formula I, LA, IB, IC, and II, n, o, p, and q are each 1.

In some embodiments of a method of using compounds of Formula I, IA, IB, IC, and II, two of n, o, p, and q are each 1 and the other two are each 0.

In some embodiments of a method of using compounds of Formula I, IA, IB, IC, and II, r is 2 and s are 6.

In some embodiments of a method of using compounds of Formula I, IA, IB, IC, and II, r is 3 and s are 5.

In some embodiments of a method of using compounds of Formula I, IA, IB, IC, and II, t is 1.

In some embodiments of a method of using compounds of Formula I, IA, IB, IC, and II, W1 and W2 are each NH, m is 0, n, and o are each 1, and p and q are each 0.

In some embodiments of a method of using compounds of Formula I, IA, IB, IC, and II, W1 and W2 are each NH, m is 1, n, o, p, and q are each 1, and L is 0.

In some embodiments of a method of using compounds of Formula I, IA, IB, IC, and II, W1 and W2 are each NH, m is 1, n, o, p, and q are each 1, and L is

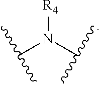

In some embodiments of a method of using compounds of Formula I, IA, IB, IC, and II, W1 and W2 are each NH, m is 1, n, o, p, and q are each 1, and L is —S—S-.

In some embodiments of a method of using compounds of Formula I, IA, IB, IC, and II, W1 and W2 are each NH, m is 1, n and o are each 0, p and q are each 1, and L is

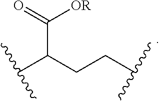

In some embodiments of a method of using compounds of Formula I, IA, IB, IC, and II, W1 and W2 are each NH, m is 1, k is 0, n and o are each 0, p and q are each 1, and L is

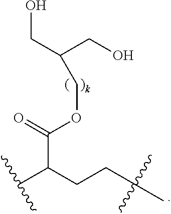

In some embodiments of a method of using compounds of Formula I, IA, IB, IC, and II, W1 and W2 are each NH, m is 1, n and o are each 1, p and q are each 0, and L is

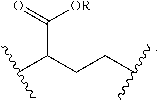

In some embodiments of a method of using compounds of Formula I, IA, IB, IC, and II, W1 and W2 are each NH, m is 1, k is 0, n is 1, o, p and q are each 0, and L is

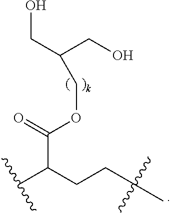

In some embodiments of a method of using compounds of Formula I, IA, IB, IC, and II, W1 and W2 are each NH, m is 1, n, o, and p are each 0, and q is 1, and L is

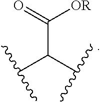

In some embodiments of a method of using compounds of Formula I, IA, IB, IC, and II, W1 and W2 are each NH, m is 1, k is 1, n, o, and p are each 0, and q is 1, and L is

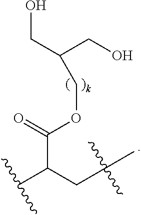

In some embodiments of a method of using compounds of Formula I, IA, IB, IC, and II, W1 and W2 are each NH, m is 1, n is 1, and o, p, and q are each 0, and L is

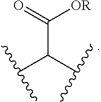

In some embodiments of a method of using compounds of Formula I, LA, IB, IC, and II, W1 and W2 are each NH, m is 1, k is 1, o, p, and q are each 0, and L is

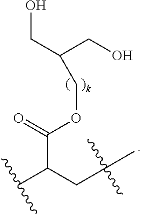

In some embodiments of a method of using compounds of Formula I, IA, IB, IC, and II, W1 and W2 are each NH, m is 1, n, o, p, and q are each 1, and L is

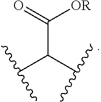

In some embodiments of a method of using compounds of Formula I, IA, IB, IC, and II, W1 and W2 are each NH, m is 1, n, o, p, and q are each 1, and L is

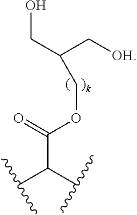

In some embodiments of a method of using compounds of Formula I, IA, IB, IC, and II, W1 and W2 are each NH, m is 0, k is 1,0 and p are each 1, and q is 0.

In some embodiments of a method of using compounds of Formula I, IA, IB, IC, and II, W1 and W2 are each NH, m is 0, n, o, p, and q are each 1.

In some embodiments of a method of using compounds of Formula I, IA, IB, IC, and II, W1 and W2 are each NH, m is 0, n and o are each 1, p and q are each 0, and each a is CH3.

In some embodiments of a method of using compounds of Formula I, I A, IB, IC, and II, W1 and W2 are each NH, m is 0, n and o are each 1, p and q are each 0, and each b is CH3.

In some embodiments of a method of using compounds of Formula I, IA, IB, IC, and II, W1 and W2 are each NH, m is 1, n, o, p, and q are each 1, R4 is H, and L is

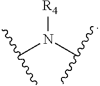

In some embodiments of a method of using compounds of Formula I, IA, IB, IC, and II, W₁ and W₂ are each NH, m is 1, n, p and q are each 1, and o is 2, R₄ is H, and L is

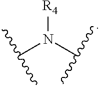

In some embodiments of a method of using compounds of Formula I, IA, IB, IC, and II, W1 and W2 are each NH, m is 1, n, o, p are each 1, and q is 2, and L is

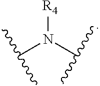

In some embodiments of a method of using compounds of Formula I, I A, IB, IC, and II, W1 and W2 are each NH, m is 1, n, o, p, and q are each 1, and L is

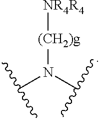

In some embodiments of a method of using compounds of Formula I, IA, IB, IC, and II, W1 and W2 are each NH, m is 1, n and p are each 1, and o and q are each 0, and L is —C(O)-.

In some embodiments of a method of using compounds of Formula I, IA, IB, IC, and II, W1 and W2 are each NH, m is 1, n and p are each 1, and 0, and q are each 0, and L is

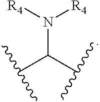

In some embodiments of a method of using compounds of Formula I, IA, IB, IC, and II, W1 and W2 are each NH, m is 1, n, o, p, q are each 1, and L is

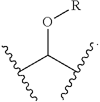

In some embodiments of a method of using compounds of Formula I, IA, IB, IC, and II, W1 and W2 are each NH, m is 1, n, o, p, and q are each 1, h is 1, and L is

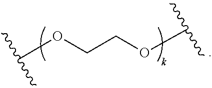

In some embodiments of a method of using compounds of Formula I, IA, IB, IC, and II, W1 and W2 are each NH, m is 1, n, o, p, and q are each 1, and L is —S-.

In some embodiments of a method of using compounds of Formula I, IA, IB, IC, and II, W1 and W2 are each NH, m is 1, n, 0, p are each 0, q is 1, one d is —CH3, and L is

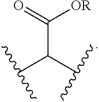

In some embodiments of a method of using compounds of Formula I, IA, IB, IC, and II, W1 and W2 are each NH, m is 2, n, o, p, and q are each 0, one L is

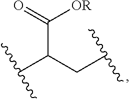

and one L is

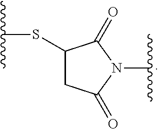 [SMILES: *SC1CC(=O)N(*)C1=O]

In some embodiments of a method of using compounds of Formula I, IA, IB, IC, and II, m is 0, n, o, p, and q are each 0, and W1 and W2 are taken together to form an optionally substituted piperazine group.

In some embodiments of a method of using compounds of Formula I, IA, IB, IC, and II, m is 1, n, o, p, and q are each 0, W1 and W2 are each null, and L is

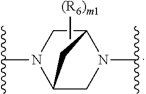

In some embodiments of a method of using compounds of Formula I, IA, IB, IC, and II, m is 1, n and p are each 1, o and q are each 0, W1 and W2 are each NH, and L is C3-C6 cycloalkyl.

In some embodiments of a method of using compounds of Formula I, IA, IB, IC, and II, m is 1, n is 1, o, p, and q are each 0, W1 and W2 are each NH, and L is C3-C6 cycloalkyl.

In some embodiments of a method of using compounds of Formula I, IA, IB, IC, and II, m is 1, n, o, p, are each 0, q is 1, W1 and W2 are each NH, and L is C3-C6 cycloalkyl.

In some embodiments of a method of using compounds of Formula I, IA, IB, IC, and II, m is 1, n, o, p, and q are each 0, W1 is NH, W2 is null, and L is

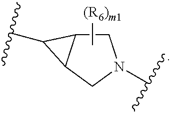

In some embodiments of a method of using compounds of Formula I, IA, IB, IC, and II, m is 1, n, o, p, and q are each 0, W1 is null, W2 is NH, and L is

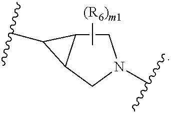

In some embodiments of a method of using compounds of Formula I, IA, IB, IC, and II, m is 1, n, o, p, and q are each 0, W1 is NH, W2 is null, and L is

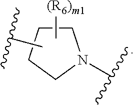

In some embodiments of a method of using compounds of Formula I, IA, IB, IC, and II, m is 1, n, o, p, and q are each 0, W1 is null, W2 is NH, and L is

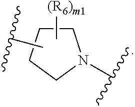

In some embodiments of method using compounds of Formula I, IA, IB, IC, and II, m is 1, n is 1, o, p, and q are each 0, W1 is NH, W2 is null, and L is

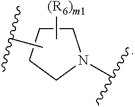

In some embodiments of a method of using compounds of Formula I, IA, IB, IC, and II, m is 1, n, o, p, are each 0, q is 1, W1 is null, W2 is NH, and L is

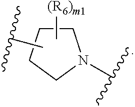

In some embodiments of a method of using compounds of Formula I, IA, IB, IC, and II, m is 1, n, o, p, and q are each 0, W1 is NH, W2 is null, and L is

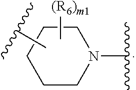

In some embodiments of a method of using compounds of Formula I, IA, IB, IC, and II, m is 1, n, o, p, and q are each 0, W1 is null, W2 is NH, and L is

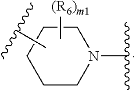

In some embodiments of a method of using compounds of Formula I, IA, IB, IC, and II, m is 1, n is 1, o, p, and q are each 0, W1 is NH, W2 is null, and L is

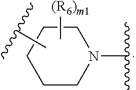

In some embodiments of a method of using compounds of Formula I, IA, IB, IC, and II, m is 1, n, o, p, are each 0, q is 1, W1 is null, W2 is NH, and L is

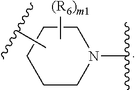

In some embodiments of a method of using compounds of Formula I, IA, IB, IC, and II, m is 1, n is 1, o, p, and q are each 0, W1 is NH, W2 is null, and L is

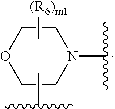

In some embodiments of a method of using compounds of Formula I, IA, IB, IC, and II, m is 1, n, o, p, are each 0, q is 1, W1 is null, W2 is NH, and L is

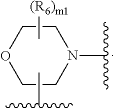

In some embodiments of a method of using compounds of Formula I, IA, IB, IC, and II, m is 1, n, o, p, q are each 0, W1 and W2 is null, and L is

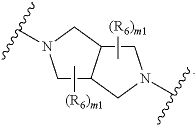

In some embodiments of a method of using compounds of Formula I, IA, IB, IC, and II, m is 1, n, o, p, q are each 0, W1 and W2 is null, and L is

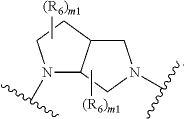

In some embodiments of a method of using compounds of Formula I, IA, IB, IC, and II, m is 1, n, o, p, q are each 0, W1 is NH, W2 is null, and L is

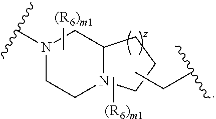

In some embodiments of a method of using compounds of Formula I, IA, IB, IC, and II, m is 1, n, o, p, q are each 0, W1 is null, W2 is NH, and L is

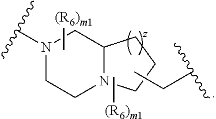

In some embodiments of a method of using compounds of Formula I, IA, IB, IC, and II, m is 1, n, o, p, are each 0, q is 1, W1 and W2 are each and NH, is null, L is

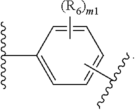

In some embodiments of a method of using compounds of Formula I, IA, IB, IC, and II, m is 1, n, o, p, are each 0, q is 1, W1 and W2 are each NH, is null, and L is a heteroaryl.

In some of the foregoing embodiments of a method of using compounds of Formula I, IA, IB, IC, and II, r is 2, s is 6 and t is 1.

In some of the foregoing embodiments of a method of using compounds of Formula I, IA, IB, IC, and II, r is 3, s is 5 and t is 1.

In some of the foregoing embodiments of a method of using compounds of Formula I, IA, IB, IC, and II, Z is

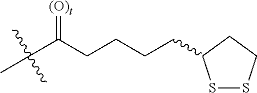

and t is 1.

In the method of using compounds of Formula I, IA, IB, IC and II, any one or more of H may be substituted with a deuterium.

In other illustrative embodiments, a method of using compounds of Formula I, IA, IB, IC and II are as set forth below:

(E)-methyl 4-(2-(4Z,7Z,10Z,13Z,16Z,19Z)-docosa-4,7,10,13,16,19-hexaenamidoethylamino)-4-oxobut-2-enoate (I-1);

(E)-methyl 4-(2-(5Z,8Z,11Z,14Z,17Z)-icosa-5,8,11,14,17-pentacnamidoethylamino)-4-oxobut-2-enoate (1-2);

(E)-methyl 4-(2-(2-(4Z,7Z,10Z,13Z,16Z,19Z)-docosa-4,7,10,13,16,19-hexaenamidoethoxy)ethylamino)-4-oxobut-2-enoate (1-3);

(E)-methyl 4-(2-((2-(4Z,7Z,10Z,13Z,16Z,19Z)-docosa-4,7,10,13,16,19-hexaenamidoethyl)(methyl)amino)ethylamino)-4-oxobut-2-enoate (1-4);

(E)-methyl 4-(2-(2-(2-(4Z,7Z,10Z,13Z,16Z,19Z)-docosa-4,7,10,13,16,19-hexaenamidoethyl)disulfanyl)ethylamino)-4-oxobut-2-enoate (1-5);

(S)-methyl 6-((4Z,7Z,10Z,13Z,16Z,19Z)-docosa-4,7,10,13,16,19-hexaenamido)-2-((E)-4-methoxy-4-oxobut-2-enamido)hexanoate (1-6);

(S)-6-((4Z,7Z,10Z,13Z,16Z,19Z)-docosa-4,7,10,13,16,19-hexaenamido)-2-((E)-4-methoxy-4-oxobut-2-enamido)hexanoic acid (1-7);

(S)-1,3-dihydroxypropan-2-yl 6-((4Z,7Z,10Z,13Z,16Z,19Z)-docosa-4,7,10,13,16,19-hexaenamido)-2-((E)-4-methoxy-4-oxobut-2- enamido)hexanoate (I-8);

(S)-methyl 2-((4Z,7Z,10Z,13Z,16Z,19Z)-docosa-4,7,10,13,16,19-hexaenamido)-6-((E)-4-methoxy-4-oxobut-2-enamido)hexanoate (I-9);

(S)-2-((4Z,7Z,10Z,13Z,16Z,19Z)-docosa-4,7,10,13,16,19-hexaenamido)-6-((E)-4-methoxy-4-oxobut-2-enamido)hexanoic acid (I-10);

(S)-1,3-dihydroxypropan-2-yl 2-((4Z,7Z,10Z,13Z,16Z,19Z)-docosa-4,7,10,13,16,19-hexaenamido)-6-((E)-4-methoxy-4-oxobut-2- enamido)hexanoate (I-11);

(E)-methyl 4-(3-(4Z,7Z,10Z,13Z,16Z,19Z)-docosa-4,7,10,13,16,19-hexaenamido-1-methoxy-1-oxopropan-2-ylamino)-4-oxobut-2-enoate (I-12);

3-(4Z,7Z,10Z,13Z,16Z,19Z)-docosa-4,7,10,13,16,19-hexaenamido-2-((E)-4-methoxy-4-oxobut-2-enamido)propanoic acid (I-13);

(E)-methyl 4-(1-(1,3-dihydroxypropan-2-yloxy)-3-(4Z,7Z,10Z,13Z,16Z,19Z)-docosa-4,7,10,13,16,19-hexaenamido-1-oxopropan-2-ylamino)-4-oxobut-2-enoate (I-14);

(E)-methyl 4-(2-(4Z,7Z,10Z,13Z,16Z,19Z)-docosa-4,7,10,13,16,19-hexaenamido-3-methoxy-3-oxopropylamino)-4-oxobut-2-enoate (I-15);

2-(4Z,7Z,10Z,13Z,16Z,19Z)-docosa-4,7,10,13,16,19-hexaenamido-3-((E)-4-methoxy-4-oxobut-2-enamido)propanoic acid (I-16);

(E)-methyl 4-(3-(1,3-dihydroxypropan-2-yloxy)-2-(4Z,7Z,10Z,13Z,16Z,19Z)-docosa-4,7,10,13,16,19-hexaenamido-3-oxopropylamino)-4-oxobut- 2-enoate (I-17);

2-(2-(4Z,7Z,10Z,13Z,16Z,19Z)-docosa-4,7,10,13,16,19-hexaenamidoethyl)-4-((E)-4-methoxy-4-oxobut-2-enamido)butanoic acid (I-18);

(E)-methyl 4-(3-(( 1,3-dihydroxypropan-2-yloxy)carbonyl)-5-(4Z,7Z,10Z,13Z,16Z,19Z)-docosa-4,7,10,13,16,19-hexaenamidopentylamino)-4-oxobut- 2-enoate (I-19);

(E)-methyl 4-(3-(4Z,7Z,10Z,13Z,16Z,19Z)-docosa-4,7,10,13,16,19-hexaenamidopropylamino)-4-oxobut-2-enoate (I-20);

(E)-methyl 4-(4-(4Z,7Z,10Z,13Z,16Z,19Z)-docosa-4,7,10,13,16,19-hexaenamidobutylamino)-4-oxobut-2-enoate (I-21);

(E)-methyl 4-(1-(4Z,7Z,10Z,13Z,16Z,19Z)-docosa-4,7,10,13,16,19-hexaenamido-2-methylpropan-2-ylamino)-4-oxobut-2-enoate (I-22);

(E)-methyl 4-(2-(4Z,7Z,10Z,13Z,16Z,19Z)-docosa-4,7,10,13,16,19-hexaenamido-2-methylpropylamino)-4-oxobut-2-enoate (I-23);

(E)-methyl 4-(2-(2-(4Z,7Z,10Z,13Z,16Z,19Z)-docosa-4,7,10,13,16,19-hexaenamidoethylamino)ethylamino)-4-oxobut-2-enoate (I-24);

(E)-methyl 4-(3-(2-(4Z,7Z,10Z,13Z,16Z,19Z)-docosa-4,7,10,13,16,19-hexaenamidoethylamino)propylamino)-4-oxobut-2-enoate (I-25);

(E)-methy 14-(2-(3-(4Z,7Z,10Z,13Z,16Z,19Z)-docosa-4,7,10,13,16,19-hexaenamidopropylamino)ethylamino)-4-oxobut-2-enoate (I-26);

(E)-methyl 4-(2-((3-(4Z,7Z,10Z,13Z,16Z,19Z)-docosa-4,7,10,13,16,19-hexaenamidopropyl)(ethyl)amino)ethylamino)-4-oxobut-2-enoate (I-27);

(E)-methyl 4-(2-(N-(3-(4Z,7Z,10Z,13Z,16Z,19Z)-docosa-4,7,10,13,16,19-hexaenamidopropyl)acetamido)ethylamino)-4-oxobut-2-enoate (I-28);

(E)-methyl 4-(2-((2-(4Z,7Z,10Z,13Z,16Z,19Z)-docosa-4,7,10,13,16,19-hexaenamidoethyl)(2-morpholinoethyl)amino)ethylamirio)-4-oxobut-2-enoate (I-29);

(E)-methyl 4-(2-((2-(4Z,7Z,10Z,13Z,16Z,19Z)-docosa-4,7,10,13,16,19-hexaenamidoethyl)(3-(piperazin-1-yl)propyl)amino)ethylamino)-4-oxobut-2-enoate (I-30);

(E)-methyl 4-(3-(4Z,7Z,10Z,13Z,16Z,19Z)-docosa-4,7,10,13,16,19-hexaenamido-2-oxopropylamino)-4-oxobut-2-enoate (I-31);

(E)-methyl 4-(3-(4Z,7Z,10Z,13Z,16Z,19Z)-docosa-4,7,10,13,16,19-hexaenamido-2-morpholinopropylamino)-4-oxobut-2-enoate (I-32);

(E)-methy 14-(3-(4Z,7Z,10Z,13Z,16Z,19Z)-docosa-4,7,10,13,16,19-hexaenamido-2-(piperazin-1-yl)propylamino)-4-oxobut-2-enoate (I-33);

(E)-methyl 4-(5-(4Z,7Z,10Z,13Z,16Z,19Z)-docosa-4,7,10,13,16,19-hexaenamido-3-hydroxypentylamino)-4-oxobut-2-enoate (I-34);

(E)-methyl 4-(5-(4Z,7Z,10Z,13Z,16Z,19Z)-docosa-4,7,10,13,16,19-hexaenamido-3-morpholinopentylamino)-4-oxobut-2-enoate (I-35);

(E)-methyl 4-(2-(2-(2-(4Z,7Z,10Z,13Z,16Z,19Z)-docosa-4,7,10,13,16,19-hexaenamidoethoxy)ethoxy)ethylamino)-4-oxobut-2-enoate (I-36);

(E)-methyl 4-(2-(2-(4Z,7Z,10Z,13Z,16Z,19Z)-docosa-4,7,10,13,16,19-hexaenamidoethylthio)ethylamino)-4-oxobut-2-enoate (I-37);

(E)-methyl 4-(3-(2-(4Z,7Z,10Z,13Z,16Z,19Z)-docosa-4,7,10,13,16,19-hexaenamidoacetoxy)-1-methoxy-1-oxobutan-2-ylamino)-4-oxobut-2-enoate (I-38);

(E)-methyl 4-((R)-3-(1-(2-(4Z,7Z,10Z,13Z,16Z,19Z)-docosa-4,7,10,13,16,19-hexaenamidoethyl)-2,5-dioxopyrrolidin-3-ylthio)-1-methoxy-1-oxopropan-2-ylamino)-4-oxobut-2-enoate (I-39);

(E)-methyl 4-(4-(4Z,7Z,10Z,13Z,16Z,19Z)-docosa-4,7,10,13,16,19-hexaenoylpiperazin-1-yl)-4-oxobut-2-enoate (I-40);

(E)-methyl 4-((2R,6S)-4-((4Z,7Z,100Z,13Z,16Z,19Z)-docosa-4,7,10,13,16,19-hexaenoyl)-2,6-dimethylpiperazin-1-yl)-4-oxobut-2- enoate (I-41);

(E)-methyl 4-((1S,4S)-5-((4Z,7Z,10Z,13Z,16Z,19Z)-docosa-4,7,10,13,16,19-hexaenoyl)-2,5-diaza-bicyclo[2.2.1]heptan-2-yl)-4-oxobut-2- enoate (I-42);

(E)-methyl 4-((2-((4Z,7Z,10Z,13Z,16Z,19Z)-docosa-4,7,10,13,16,19-hexaenamidomethyl)cyclopropyl)methylamino)-4 oxobut-2-enoate (I-43);

(E)-methyl 4-((4-(4Z,7Z,10Z,13Z,16Z,19Z)-docosa-4,7,10,13,16,19-hexaenamidocyclohexyl)methylamino)4-oxobut-2-enoate (I-44);

(E)-methy 14-(4-((4Z,7Z,10Z,13Z,16Z,19Z)-docosa-4,7,10,13,16,19-hexaenamidomethyl)cyclohexylamino)-4-oxobut-2-enoate (I-45);

(E)-methyl 4-(3-(4Z,7Z,10Z,13Z,16Z,19Z)-docosa-4,7,10,13,16,19-hexaenoyl-3-aza-bicyclo[3.1.0]hexan-6-ylamino)-4-oxobut-2-enoate (I-46);

(E)-methyl 4-(6-(4Z,7Z,10Z,13Z,16Z,19Z)-docosa-4,7,10,13,16,19-hexaenamido-3-aza-bicyclo[3.1.0]hexan-3-yl)-4-oxobut-2-enoate (I-47);

(E)-methyl 4-((S)-1-((4Z,7Z,10Z,13Z,16Z,19Z)-docosa-4,7,10,13,16,19-hexaenoyl)pyrrolidin-3-ylamino)-4-oxobut-2-enoate (I-48);

(E)-methy 14-((S)-3-((4Z,7Z,10Z,13Z,16Z,19Z)-docosa-4,7,10,13,16,19-hexaenamido)pyrrolidin-1-yl)-4-oxobut-2-enoate (I-49);

(E)-methyl 4-((1-(4Z,7Z,10Z,13Z,16Z,19Z)-docosa-4,7,10,13,16,19-hexaenoylpyrrolidin-2-yl)methylamino)-4-oxobut-2-enoate (I-50);

(E)-methyl 4-(2-((4Z,7Z,10Z,13Z,16Z,19Z)-docosa-4,7,10,13,16,19-hexaenamidomethyl)pyrrolidin-1-yl)-4-oxobut-2-enoate (I-51);

(E)-methyl 4-(1-(4Z,7Z,10Z,13Z,16Z,19Z)-docosa-4,7,10,13,16,19-hexaenoylpiperidin-4-ylamino)-4-oxobut-2-enoate (I-52);

(E)-methyl 4-(4-(4Z,7Z,10Z,13Z,16Z,19Z)-docosa-4,7,10,13,16,19-hexaenamidopiperidin-1-yl)-4-oxobut-2-enoate (I-53);

(E)-methyl 4-((1-(4Z,7Z,10Z,13Z,16Z,19Z)-docosa-4,7,10,13,16,19-hexaenoylpiperidin-4-yl)methylamino)-4-oxobut-2-enoate (I-54);

(E)-methyl 4-(4-((4Z,7Z,10Z,13Z,16Z,19Z)-docosa-4,7,10,13,16,19-hexaenamidomethyl)piperidin-1-yl)-4-oxobut-2-enoate (I-55);

(E)-methyl 4-((1-(4Z,7Z,10Z,13Z,16Z,19Z)-docosa-4,7,10,13,16,19-hexaenoylpiperidin-2-yl)methylamino)-4-oxobut-2-enoate (I-56);

(E)-methyl 4-(2-((4Z,7Z,10Z,13Z,16Z,19Z)-docosa-4,7,10,13,16,19-hexaenamidomethyl)piperidin-1-yl)-4-oxobut-2-enoate (I-57);

(E)-methyl 4-((4-(4Z,7Z,10Z,13Z,16Z,19Z)-docosa-4,7,10,13,16,19-hexaenoylmorpholin-3-yl)methylamino)-4-oxobut-2-enoate (I-58);

(E)-methyl 4-(3-((4Z,7Z,10Z,13Z,16Z,19Z)-docosa-4,7,10,13,16,19-hexaenamidomethyl)morpholino)-4-oxobut-2-enoate (I-59);

(E)-methyl 4-(5-(4Z,7Z,10Z,13Z,16Z,19Z)-docosa-4,7,10,13,16,19-hexaenoyl-hexahydropyrrolo[3,4-c]pyrrol-2(1H)-yl)-4-oxobut-2-enoate (I-60);

(E)-methyl 4-(1-(4Z,7Z,10Z,13Z,16Z,19Z)-docosa-4,7,10,13,16,19-hexaenoyl-hexahydropyrrolo[3,4-b]pyrrol-5(1H)-yl)-4-oxobut-2-enoate (I-61);

(E)-methyl 4-((2-(4Z,7Z,10Z,13Z,16Z,19Z)-docosa-4,7,10,13,16,19-hexaenoyloctahydropyrrolo[1,2-a]pyrazin-7-yl)methylamino)-4-oxobut-2- enoate (I-62);

(E)-methyl 4-(7-((4Z,7Z,10Z,13Z,16Z,19Z)-docosa-4,7,10,13,16,19-hexaenamidomethyl)-hexahydropyrrolo[1,2-a]pyrazin-2(1H)-yl)-4-oxobut-2-enoate (I-63);

(E)-methyl 4-(4-((4Z,7Z,10Z,13Z,16Z,19Z)-docosa-4,7,10,13,16,19-hexaenamidomethyl)phenylamino)-4-oxobut-2-enoate (I-64);

(E)-methyl 4-(6-((4Z,7Z,10Z,13Z,16Z,19Z)-docosa-4,7,10,13,16,19-hexaenamidomethyl)pyridin-2-ylamino)-4-oxobut-2-enoate (I-65);

(E)-ethyl 4-(2-(4Z,7Z,10Z,13Z,16Z,19Z)-docosa-4,7,10,13,16,19-hexaenamidoethylamino)-4-oxobut-2-enoate (I-66);

(E)-ethyl 4-(2-((2-(4Z,7Z,10Z,13Z,16Z,19Z)-docosa-4,7,10,13,16,19-hexaenamidoethyl)(methyl)amino)ethylamino)-4-oxobut-2-enoate (I-67);

(E)-ethyl 4-(2-(2-(4Z,7Z,10Z,13Z,16Z,19Z)-docosa-4,7,10,13,16,19-hexaenamidoethylamino)ethylamino)-4-oxobut-2-enoate (I-68);

(S)-6-((4Z,7Z,10OZ, 13Z, 16Z, 19Z)-docosa-4,7,10,13,16,19-hexaenamido)-2-((E)-4-ethoxy-4-oxobut-2-enamido)hexanoic acid (1-69);

(S)-2-((4Z,7Z,10Z,13Z,16Z,19Z)-docosa-4,7,10,13,16,19-hexaenamido)-6-((E)-4-ethoxy-4-oxobut-2-enamido)hexanoic acid (I-70);

(S)-6-((5Z,8Z,11Z,14Z,17Z)-icosa-5,8,11,14,17-pentaenamido)-2-((E)-4-methoxy-4-oxobut-2-enamido)hexanoic acid (I-71);

(S)-2-((5Z,8Z,11Z,14Z,17Z)-icosa-5,8,11,14,17-pentaenamido)-6-((E)-4-methoxy-4-oxobut-2-enamido)hexanoic acid (I-72);

(E)-methyl 4-(2-(2-(5Z,8Z,11Z,14Z,17Z)-icosa-5,8,11,14,17-pentaenamidoethylamino)ethylamino)-4-oxobut-2-enoate (I-73);

(E)-methyl 4-(2-((2-(5Z,8Z,11Z,14Z,17Z)-icosa-5,8,11,14,17-pentaenamidoethyl)(methyl)amino)ethylamino)-4-oxobut-2-enoate (I-74);

(E)-ethyl 4-(2-(5Z,8Z,11Z,14Z,17Z)-icosa-5,8,11,14,17-pentaenamidoethylamino)-4-oxobut-2-enoate (I-75);

(S)-2-((E)-4-ethoxy-4-oxobut-2-enamido)-6-((5Z,8Z,11Z,14Z,17Z)-icosa-5,8,11,14,17-pentaenamido)hexanoic acid (I-76);

(S)-6-((E)-4-ethoxy-4-oxobut-2-enamido)-2-((5Z,8Z,11Z,14Z,17Z)-icosa-5,8,11,14,17-pentaenamido)hexanoic acid (I-77);

(E)-ethyl 4-(2-((2-(5Z,8Z,11Z,14Z,17Z)-icosa-5,8,11,14,17-pentaenamidoethyl)(methyl)amino)ethylamino)-4-oxobut-2-enoate (I-78);

(E)-ethyl 4-(2-(2-(5Z,8Z,11Z,14Z,17Z)-icosa-5,8,11,14,17-pentaenamidoethylamino)ethylamino)-4-oxobut-2-enoate (I-79);

(S)-5-((4Z,7Z,10Z,13Z,16Z,19Z)-docosa-4,7,10,13,16,19-hexaenamido)-2-((E)-4-methoxy-4-oxobut-2-enamido)pentanoic acid (I-80);

(S)-2-((4Z,7Z,10Z,13Z,16Z,19Z)-docosa-4,7,10,13,16,19-hexaenamido)-5-((E)-4-methoxy-4-oxobut-2-enamido)pentanoic acid (I-81);

(S)-1,3-dihydroxypropan-2-yl 5-((4Z,7Z,10Z,13Z,16Z,19Z)-docosa-4,7,10,13,16,19-hexaenamido)-2-((E)-4-methoxy-4-oxobut-2- enamido)pentanoate (I-82);

(S)-1,3-dihydroxypropan-2-yl 2-((4Z,7Z,10Z,13Z,16Z,19Z)-docosa-4,7,10,13,16,19-hexaenamido)-5-((E)-4-methoxy-4-oxobut-2- enamido)pentanoate (I-83);

(S)-5-((5Z,8Z,11Z,14Z,17Z)-icosa-5,8,11,14,17-pentaenamido)-2-((E)-4-methoxy-4-oxobut-2-enamido)pentanoic acid (I-84);

(S)-2-((5Z,8Z,11Z,14Z,17Z)-icosa-5,8,11,14,17-pentaenamido)-5-((E)-4-methoxy-4-oxobut-2-enamido)pentanoic acid (I-85);

(S)-1,3-dihydroxypropan-2-yl 5-((5Z,8Z,11Z,14Z,17Z)-icosa-5,8,11,14,17-pentaenamido)-2-((E)-4-methoxy-4-oxobut-2-enamido)pentanoate (I-86);

(S)-1,3-dihydroxypropan-2-yl 2-((5Z,8Z,11Z,14Z,17Z)-icosa-5,8,11,14,17-pentaenamido)-5-((E)-4-methoxy-4-oxobut-2-enamido)pentanoate (I-87);

(S)-5-((4Z,7Z,10Z,13Z,16Z,19Z)-docosa-4,7,10,13,16,19-hexaenamido)-2-((E)-4-ethoxy-4-oxobut-2-enamido)pentanoic acid (I-88);

(S)-2-((4Z,7Z,10Z,13Z,16Z,19Z)-docosa-4,7,10,13,16,19-hexaenamido)-5-((E)-4-ethoxy-4-oxobut-2-enamido)pentanoic acid (I-89);

(S)-1,3-dihydroxypropan-2-yl 5-((4Z,7Z,10Z,13Z,16Z,19Z)-docosa-4,7,10,13,16,19-hexaenamido)-2-((E)-4-ethoxy-4-oxobut-2- enamido)pentanoate (I-90);

(S)-1,3-dihydroxypropan-2-yl 2-((4Z,7Z,10Z,13Z,16Z,19Z)-docosa-4,7,10,13,16,19-hexaenamido)-5-((E)-4-ethoxy-4-oxobut-2- enamido)pentanoate (I-91);

(S)-2-((E)-4-ethoxy-4-oxobut-2-enamido)-5-((5Z,8Z,11Z,14Z,17Z)-icosa-5,8,11,14,17-pentaenamido)pentanoic acid (I-92);

(S)-5-((E)-4-ethoxy-4-oxobut-2-enamido)-2-((5Z,8Z,11Z,14Z,17Z)-icosa-5,8,11,14,17-pentaenamido)pentanoic acid (I-93);

(S)-1,3-dihydroxypropan-2-yl 2-((E)-4-ethoxy-4-oxobut-2-enamido)-5-((5Z,8Z,11Z,14Z,17Z)-icosa-5,8,11,14,17-pentaenamido)pentanoate (I-94);

(S)-1,3-dihydroxypropan-2-yl 5-((E)-4-ethoxy-4-oxobut-2-enamido)-2-((5Z,8Z,11Z,14Z,17Z)-icosa-5,8,11,14,17-pentaenamido)pentanoate (I-95);

(S)-1,3-dihydroxypropan-2-yl 6-((5Z,8Z,11Z,14Z,17Z)-icosa-5,8,11,14,17-pentaenamido)-2-((E)-4-methoxy-4-oxobut-2-enamido)hexanoate (I-96);

(S)-1,3-dihydroxypropan-2-yl 2-((5Z,8Z,11Z,14Z,17Z)-icosa-5,8,11,14,17-pentaenamido)-6-((E)-4-methoxy-4-oxobut-2-enamido)hexanoate (I-97);

(S)-1,3-dihydroxypropan-2-yl 6-((4Z,7Z,10Z,13Z,16Z,19Z)-docosa-4,7,10,13,16,19-hexacnamido)-2-((E)-4-ethoxy-4-oxobut-2- enamido)hexanoate (I-98);

(S)-1,3-dihydroxypropan-2-yl 2-((E)-4-ethoxy-4-oxobut-2-enamido)-6-((5Z,8Z,11Z,14Z,17Z)-icosa-5,8,11,14,17-pentaenamido)hexanoate (I-99);

(S)-1,3-dihydroxypropan-2-yl 2-((4Z,7Z,10Z,13Z,16Z,19Z)-docosa-4,7,10,13,16,19-hexaenamido)-6-((E)-4-ethoxy-4-oxobut-2- enamido)hexanoate (I-100);

(S)-1,3-dihydroxypropan-2-yl 6-((E)-4-ethoxy-4-oxobut-2-enamido)-2-((5Z,8Z,11Z,14Z,17Z)-icosa-5,8,11,14,17-pentaenamido)hexanoate (I-101);

(E)-4-(2-(4Z,7Z,10Z,13Z,16Z,19Z)-docosa-4,7,10,13,16,19-hexaenamidoethylamino)-4-oxobut-2-enoic acid (I-102);

2-(4Z,7Z,10Z,13Z,16Z,19Z)-docosa-4,7,10,13,16,19-hexaenamidoethyl methyl fumarate (I-103);

(E)-methyl 4-(methyl(2-((4Z,7Z,10Z,13Z,16Z,19Z)-N-methyldocosa-4,7,10,13,16,19-hexaenamido)ethyl)amino)-4-oxobut-2-enoate (I-104);

(R,E)-methyl 4-(2-(5-(1,2-dithiolan-3-yl)pentanamido)ethylamino)-4-oxobut-2-enoate (I-105);

6-(5-((R)-1,2-dithiolan-3-yl)pentanamido)-2-((E)-4-methoxy-4-oxobut-2-enamido)hexanoic acid (I-106);

2-(5-((R)-1,2-dithiolan-3-yl)pentanamido)-6-((E)-4-methoxy-4-oxobut-2-enamido)hexanoic acid (I-107);

(R,E)-methyl 4-(2-(2-(5-(1,2-dithiolan-3-yl)pentanamido)ethylamino)ethylamino)-4-oxobut-2-enoate (I-108);

(R,E)-methyl 4-(2-((2-(5-(1,2-dithiolan-3-yl)pentanamido)ethyl)(methyl)amino)ethylamino)-4-oxobut-2-enoate (I-109);

N1, N4-bis(2-(4Z,7Z,10Z,13Z,16Z,19Z)-docosa-4,7,10,13,16,19-hexaenamidoethyl)fumaramide (II-1); and

N1-(2-(4Z,7Z,10Z,13Z,16Z,19Z)-docosa-4,7,10,13,16,19-hexaenamidoethyl)-N4-(2-(5Z,8Z,11Z,14Z,17Z)-icosa-5,8,11,14,17-pentaenamidoethyl)fumaramide (II-2).

### Methods of Making Fatty Acid Fumarate Derivative

FAFDs of Formula Formula I, IA, IB, IC and II disclosed herein may be obtained via the synthetic methods described in U.S. Pat. No. 8,969,354, the entire disclosure of which is incorporated herein by reference. General synthetic methods useful in the synthesis of compounds described herein are available in the art.

### Pharmaceutical Composition

The invention also includes pharmaceutical compositions useful for treating or preventing a heart failure disease, including HFPEF. The compositions may be suitable for internal use and comprise an effective amount of a FAFDand a pharmaceutically acceptable carrier. The FAFDs are especially useful in that they demonstrate very low peripheral toxicity or no peripheral toxicity.

Administration of the FAFDs can be accomplished via any mode of administration for therapeutic agents. These modes include systemic or local administration such as oral, nasal, parenteral, transdermal, subcutaneous, vaginal, buccal, rectal or topical administration modes.

Depending on the intended mode of administration, the compositions can be in a solid, semi-solid, or liquid dosage form, such as, for example, injectables, tablets, suppositories, pills, time-release capsules, elixirs, tinctures, emulsions, syrups, powders, liquids, suspensions, or the like, sometimes in unit dosages and consistent with conventional pharmaceutical practices. Likewise, they can also be administered in intravenous (both bolus and infusion), intraperitoneal, subcutaneous or intramuscular form, all using forms well known to those skilled in the pharmaceutical arts.

Illustrative pharmaceutical compositions are tablets and gelatin capsules comprising a FAFD and a pharmaceutically acceptable carrier, such as a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, algiic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl-cyclodextrin, PEG400, PEG200.

Liquid, particularly injectable, compositions can, for example, be prepared by dissolution, dispersion, etc. For example, the FAFD is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the FAFDs.

The FAFDs can be also formulated as a suppository that can be prepared from fatty emulsions or suspensions, doe example using polyalkylene glycols such as propylene glycol, as the carrier.

The FAFDs can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564, the entire disclosure of which is herein incorporated by reference.

FAFDs can also be delivered by the use of monoclonal antibodies as individual carriers to which the FAFDs are coupled. The FAFDs can also be coupled with soluble polymers as targetable drug carriers. Such polymers can include polyvinylpyrrolidone, pyran copolymer, polyhydroxypropylmethacrylamide-phenol, polyhydroxyethylaspanamidephenol, or polyethyleneoxidepolylysine substituted with palmitoyl residues. Furthermore, the FAFDs can be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels. In one embodiment, FAFDs are not covalently bound to a polymer, e.g., a polycarboxylic acid polymer, or a polyacrylate.

Parenteral injectable administration is generally used for subcutaneous, intramuscular, or intravenous injections and infusions. Injectables can be prepared in conventional forms, either as liquid solutions or suspensions or solid forms suitable for dissolving in liquid prior to injection.

Compositions can be prepared according to conventional mixing, granulating, or coating methods, respectively, and the present pharmaceutical compositions can contain from about 0.1% to about 80%, from about 5% to about 60%, or from about 1% to about 20% of the FAFD by weight or volume.

### Methods Of Using Fatty Acid Fumarate Derivatives

Also provided in the present disclosure is a method for inhibiting, preventing, or treating a heart failure disease in a subject by administration of a FAFD of Formula I, IA, IB, IC, and II.

For example, the heart failure disease is heart failure with preserved ejection fraction (HFPEF).

For example, the heart failure disease is heart failure with an ejection fraction ≧40%.

For example, the heart failure disease is diastolic heart failure.

For example, the heart failure disease is heart failure with increased levels of TNF-α, IL-6, CRP, or other pro-inflammatory cytokines

For example, the heart failure disease is hypertension with risk of developing HFPEF.

For example, the heart failure disease is atrial fibrillation with risk of developing HFPEF.

For example, the heart failure disease is diabetes with risk of developing HFPEF.

For example, the heart failure disease is COPD with risk of developing HFPEF.

For example, the heart failure disease is ischemic heart disease with risk of developing HFPEF.

For example, the heart failure disease is obesity with risk of developing HFPEF.

For example, the heart failure disease is chronic heart failure.

For example, the heart failure disease is compensated heart failure.

For example, the heart failure disease is decompensated heart failure.

For example, the heart failure disease is a condition that has a high risk of developing HFPEF.

In one embodiment, the present disclosure provides a method of treating a heart failure disease in a subject in need thereof, the method comprising administering to the subject a therapeutically effective amount of one or more FAFDs of Formula I

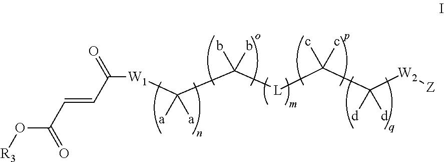

or a pharmaceutically acceptable salt, hydrate, enantiomer, or stereoisomer thereof;

wherein

each W1 and W2 is independently null, O, S, NH, or NR, or W1 and W2 can be taken together to form an optionally substituted imidazolidine or piperazine group;

each a, b, c, and d, is independently —H, —D, —CH3, —OCH3, —OCH2CH3, —C(O)OR, —O—Z, or benzyl, or two of a, b, c, and d can be taken together, along with the single carbon to which they are bound, to form a cycloalkyl or heterocycle;

each n, o, p, and q is independently 0, 1, or 2;

each L is independently null, —O—, —C(O)—, —S—, —S(O)—, —S(O)2—, —S—S—, —(C1-C6 alkyl)-, —(C3-C6 cycloalkyl)-, a heterocycle, a heteroaryl,

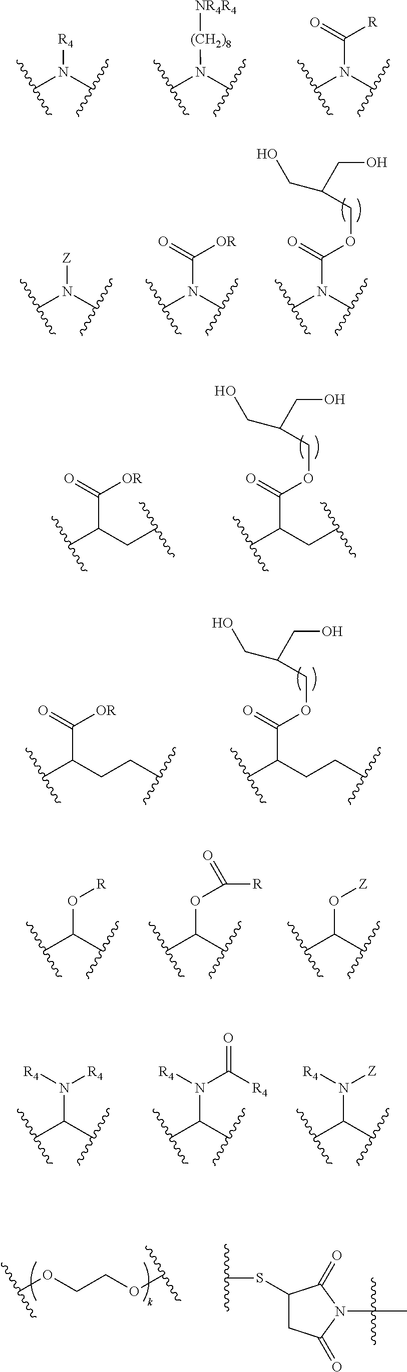

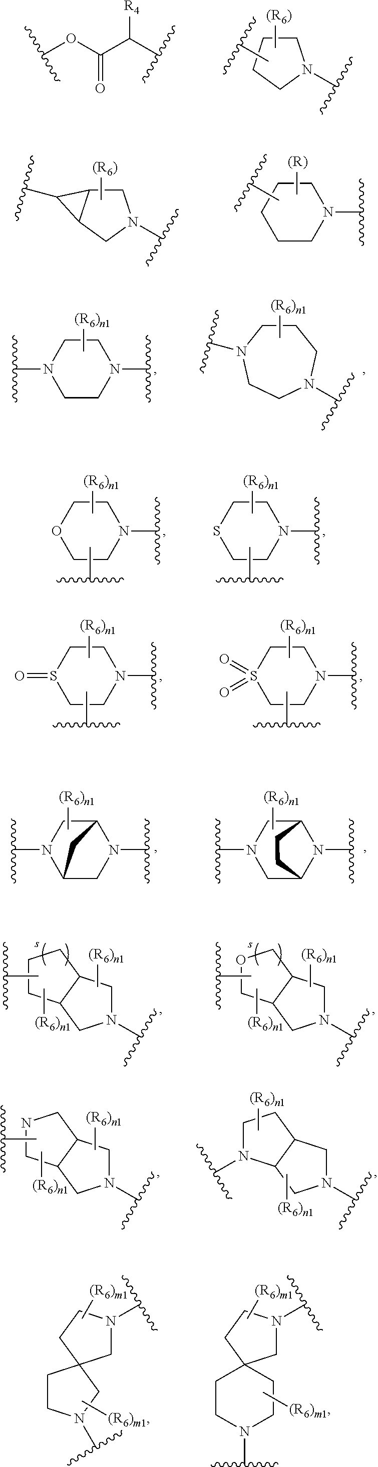

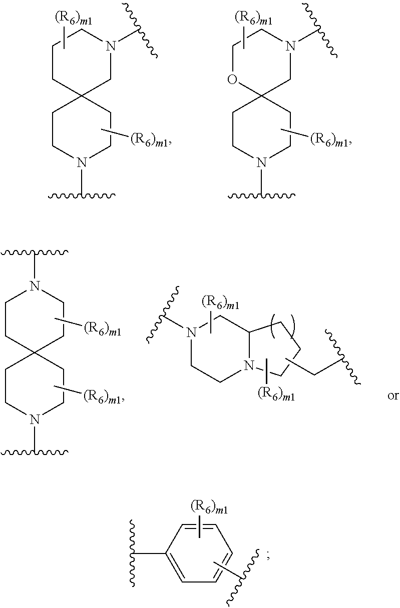

wherein the representation of L is not limited directionally left to right as is depicted, rather either the left side or the right side of L can be bound to the W₁ side of the compound of Formula I;

each is independently —H, —D, —C₁-C₄ alkyl, -halogen, cyano, oxo, thiooxo, —OH, —C(O)C₁-C₄ alkyl, —O-aryl, —O-benzyl, —OC(O)C₁-C₄ alkyl, —C₂-C₃ alkene, —C₂-C₃ alkyne, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, or —S(O)₂C₁-C₃ alkyl;

each g is independently 2, 3, or 4;

each h is independently 1,2,3, or 4;

each m is independently 0,1,2, or 3; if m is more than 1, then L can be the same or different;

each ml is independently 0,1, 2, or 3;

k is 0, 1, 2, or 3;

z is 1, 2, or 3;

each R₄ is independently H or optionally substituted C₁-C₆ alkyl, wherein a methylene unit of the C₁-C₆ alkyl can be optionally substituted for either O or NR, and in NR₄R₄, both R₄ when taken together with the nitrogen to which they are attached can form a heterocyclic ring such as a pyrrolidine, piperidine, morpholine, piperazine or pyrrole;

each Z is independently H,

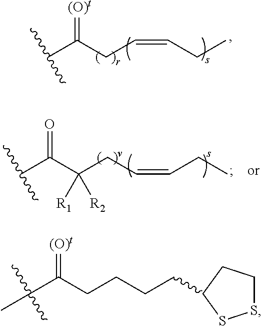

provided that there is at least one

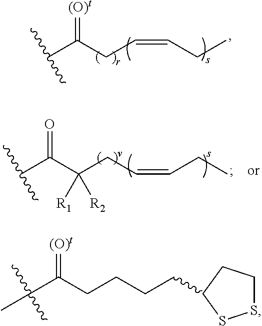

in the compound;

each t is independently 0 or 1;

each r is independently 2, 3, or 7;

each s is independently 3, 5, or 6;

each v is independently 1,2, or 6;

each R₁ and R₂ is independently —H, —D, —C₁-C₄ alkyl, -halogen, —OH, —C(O)C₁-C₄ alkyl, —O-aryl, —O-benzyl, —OC(O)C₁-C₄ alkyl, —C₂-C₃ alkene, —C₂-C₃ alkyne, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, or —S(O)₂C₁-C₃ alkyl;

each R₃ is independently H, or —C₁-C₆ alkyl;

each R₅ is independently e, H, or straight or branched C₁-C₁₀ alkyl which can be optionally substituted with OH, NH₂, CO₂R, CONH₂, phenyl, C₆H₄OH, imidazole or arginine;

each e is independently H or any one of the side chains of the naturally occurring amino acids;

each R is independently —H, —C₁-C₃ alkyl, or straight or branched C₁-C₄ alkyl optionally substituted with OH, or halogen;

provided that when each of m, n, o, p, and q is 0, W₁ and W₂ are each null, and Z is

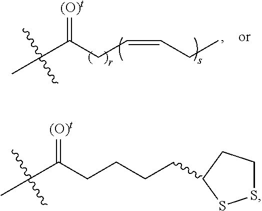

then t must be 0; and

when each of m, n, o, p, and q is 0, and W₁ and W₂ are each null, then Z must not be

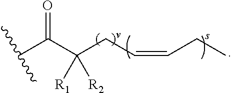

In one embodiment, the present disclosure provides method of treating a heart failure disease in a subject in need thereof, the method comprising administering to the subject a therapeutically effective amount of FAFD of formula

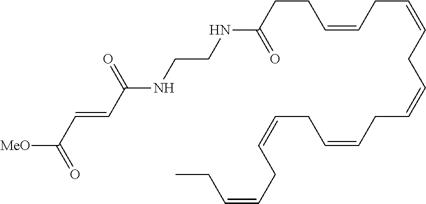 [SMILES: CC/C=C\C/C=C\C/C=C\C/C=C\C/C=C\C/C=C\CCC(=O)NCCNC(=O)/C=C/C(=O)OC]

(E)-methyl 4-(2-(4Z,7Z,10Z,13Z,16Z,19Z)-docosa-4,7,10,13,16,19-hexaenamidoethylamino)-4-oxobut-2-enoate (I-1) or a pharmaceutically acceptable salt thereof.

In certain embodiments, the heart failure disease may be heart failure with preserved ejection fraction (HFPEF); heart failure with ejection fraction ≧40%; diastolic heart failure; heart failure with elevated levels of TNF-α, IL-6, CRP, or TGF-β; hypertension with a risk of developing HFPEF; atrial fibrillation with a risk of developing HFPEF; diabetes with a risk of developing HFPEF; COPD with a risk of developing HFPEF; ischemic heart disease with a risk of developing HFPEF; obesity with a risk of developing HFPEF; chronic heart failure; compensated heart failure; decompensated heart failure; or other conditions known to have a high risk of developing HFPEF. In particular, heart failure disease is heart failure with preserved ejection fraction (HFPEF).

FAFDs may be assayed in vitro and in vivo for the desired therapeutic or prophylactic activity prior to use in humans. In vivo assays, for example using appropriate animal models, may also be used to determine whether administration of a FAFD is therapeutically effective.

### Dosage and Adminstration

In some embodiments, the subject is administered an effective amount of a FAFD.

The dosage regimen utilizing the FAFD is selected in accordance with a variety of factors including type, species, age, weight, sex and medical condition of the subject; the severity of the condition to be treated; the route of administration; the renal or hepatic function of the subject; and the particular FAFD employed. A physician or veterinarian of ordinary skill in the art can readily determine and prescribe the effective amount of the drug required to prevent, counter, or arrest the progress of the condition.

In addition, in vitro or in vivo assays may be employed to help identify optimal dosage ranges. For systemic administration, a therapeutically effective dose may be estimated initially from in vitro assays. For example, a dose may be formulated in animal models to achieve a beneficial circulating composition concentration range. Initial doses may also be estimated from in vivo data, e.g., animal models, using techniques that are known in the art. Such information may be used to more accurately determine useful doses in humans. One having ordinary skill in the art may optimize administration to humans based on animal data.

Effective dosage amounts of the present invention, when used for the indicated effects, range from about 20 mg to about 5000 mg of the FAFD per day. Compositions for in vivo or in vitro use can contain about 20, 50, 75,100, 150,250, 500, 750, 1000, 1250, 2500, 3500, or 5000 mg of the FAFD. In one embodiment, the composition is in the form of a tablet that can be scored. Effective plasma levels of the FAFD can range from about 0.002 mg to about 100 mg per kg of body weight per day. Appropriate dosages of the FAFDs can be determined as set forth in Goodman, L. S.; Gilman, A. The Pharmacological Basis of Therapeutics, 5th ed.; MacMillan: New York, 1975, pp. 201-226.

FAFDs can be administered in a single daily dose, or the total daily dosage can be administered in divided doses of two, three or four times daily. Furthermore, FAFDs can be administered in intranasal form via topical use of suitable intranasal vehicles, or via transdermal routes, using those forms of transdermal skin patches well known to those of ordinary skill in that art. To be administered in the form of a transdermal delivery system, the dosage administration can be continuous rather than intermittent throughout the dosage regimen. Other illustrative topical preparations include creams, ointments, lotions, aerosol sprays, and gels, wherein the concentration of the FAFD ranges from about 0.1% to about 15%, w/w or w/v.

In certain embodiments, a therapeutically effective dose of a FAFD may provide therapeutic benefit without causing substantial toxicity including adverse side effects. Toxicity of FAFD and/or metabolites thereof may be determined using standard pharmaceutical procedures and may be ascertained by those skilled in the art. The dose ratio between toxic and therapeutic effect is the therapeutic index. A dose of a FAFD may be within a range capable of establishing and maintaining a therapeutically effective circulating plasma and/or blood concentration of a FAFD that exhibits little or no toxicity. A dose may vary within this range depending upon the dosage form employed and the route of administration utilized. In certain embodiments, an escalating dose may be administered.

The dose will be adjusted to the individual requirements in each particular case. That dosage may vary within wide limits depending upon numerous factors such as the severity of the disease to be treated, the age and general health condition of the subject, other medicaments with which the subject is being treated, the route and form of administration, and the preferences and experience of the medical practitioner involved.

For oral administration, therapeutically effective amount of

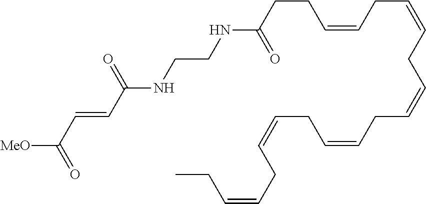 [SMILES: CC/C=C\C/C=C\C/C=C\C/C=C\C/C=C\C/C=C\CCC(=O)NCCNC(=O)/C=C/C(=O)OC]

(E)-methyl 4-(2-(4Z,7Z,10Z,13Z,16Z,19Z)-docosa-4,7,10,13,16,19-hexaenamidoethylamino)-4-oxobut-2-enoate (I-1) or a pharmaceutically acceptable salt thereof that is shown to provide MMF plasma exposure comparable to dimethyl fumarate (DMF) 120 mg to 720 mg per day as a monotherapy and/or in combination therapy.

In one embodiment, daily dose comprises about 20 mg to about 5000 mg of

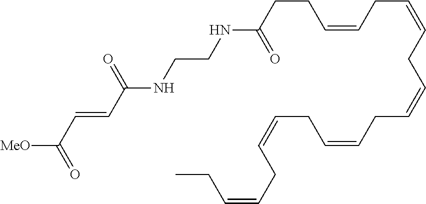 [SMILES: CC/C=C\C/C=C\C/C=C\C/C=C\C/C=C\C/C=C\CCC(=O)NCCNC(=O)/C=C/C(=O)OC]

(E)-methyl 4-(2-(4Z,7Z,10Z,13Z,16Z,19Z)-docosa-4,7,10,13,16,19-hexaenamidoethylamino)-4-oxobut-2-enoate or a pharmaceutically acceptable salt thereof.

One of ordinary skill in treating diseases described herein will be able, without undue experimentation and in reliance on personal knowledge, experience and the disclosures of this application, to ascertain a therapeutically effective amount of the compounds of the present invention for a given disease and subject.

### Combination Therapy

Methods provided by the present disclosure further comprise administering one or more pharmaceutically active compounds in addition to one or more a FAFDs.

Such compounds may be provided to treat the same disease or a different disease than the disease being treated with the FAFDs.

In certain embodiments, FAFDs may be used in combination with at least one other therapeutic agent. In certain embodiments, FAFDs may be administered to a subject together with another compound for treating a heart failure disease, such as HFPEF.

FAFDs and the at least one other therapeutic agent may act additively or, and in certain embodiments, synergistically. The at least one additional therapeutic agent may be included in the same dosage form as FAFDs or may be provided in a separate dosage form. Methods provided by the present disclosure can further include, in addition to administering a FAFD, administering one or more therapeutic agents effective for treating the same or different disease than the disease being treated by a FAFDs. Methods provided by the present disclosure include administration of a FAFD and one or more other therapeutic agents provided that the combined administration does not inhibit the therapeutic efficacy of the FAFD and/or does not typically produce significant and/or substantial adverse combination effects.

In certain embodiments, dosage forms comprising FAFDs may be administered concurrently with the administration of another therapeutic agent, which may be part of the same dosage form as, or in a different dosage form than that comprising a FAFD.

A FAFD may be administered prior or subsequent to administration of another therapeutic agent. In certain embodiments of combination therapy, the combination therapy may comprise alternating between administering a FAFD and a composition comprising another therapeutic agent, e.g., to minimize adverse drug effects associated with a particular drug. When a FAFD is administered concurrently with another therapeutic agent that potentially may produce an adverse drug effect including, but not limited to, toxicity, the other therapeutic agent may advantageously be administered at a dose that falls below the threshold at which the adverse drug reaction is elicited.

In certain embodiments, dosage forms comprising a FAFD maybe administered with one or more substances to enhance, modulate and/or control release, bioavailability, therapeutic efficacy, therapeutic potency, stability, and the like of a FAFD. For example, to enhance the therapeutic efficacy of a FAFD, the FAFD may be co-administered with or a dosage form comprising a FAFD may comprise one or more active agents to increase the absorption or diffusion of a FAFD from the gastrointestinal tract to the systemic circulation, or to inhibit degradation of the FAFD in the blood of a subject. In certain embodiments, a FAFD may be co-administered with an active agent having pharmacological effects that enhance the therapeutic efficacy of a FAFD.

In certain embodiments, FAFDs provided by the present disclosure and pharmaceutical compositions thereof may be administered to a subject for treating heart failure with preserved ejection fraction (HFPEF) in combination with a therapy or another therapeutic agent known or believed to be effective in treating HFPEF.

In certain embodiments, administration of FAFD may also be carried out in the combination with administration of one or more preparations of a second agent useful for treating heart failure, such as but not limited to diuretics, ace-inhibitors, beta-blockers, angiotensin receptor blockers, isosorbide dinitrate, hydralazine , angiotensin receptor-neprilysin inhibitors, aldosterone antagonists, a PDE5 inhibitor, a statin, a neprilysin inhibitor, an aldosterone inhibitor, or an antitumor necrosis factor-alpha therapy. In one embodiment, the second agent is a statin, for example atorvastatin, fluvastatin, lovastatin, pravastatin, rosuvastatin, or simvastatin. For this purpose, the preparations administered may comprise a combination of the active ingredients in the known dosages or amounts, respectively.

In certain embodiments, FAFDs provided by the present disclosure and pharmaceutical compositions thereof may be administered to a subject for treating heart failure with preserved ejection fraction (HFPEF) in combination with a statin (HMG-CoA reductase inhibitor).

In one embodiment, combination relates to (a)

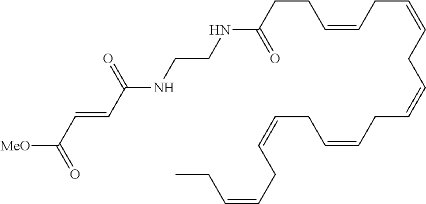 [SMILES: CC/C=C\C/C=C\C/C=C\C/C=C\C/C=C\C/C=C\CCC(=O)NCCNC(=O)/C=C/C(=O)OC]

(E)-methyl 4-(2-(4Z,7Z,10Z,13Z,16Z,19Z)-docosa-4,7,10,13,16,19-hexaenamidoethylamino)-4-oxobut-2-enoate or a pharmaceutically acceptable salt thereof and (b) a statin.

In some embodiments, a pharmaceutical composition is provided comprising

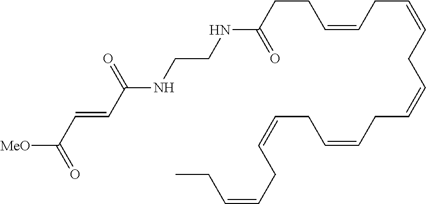 [SMILES: CC/C=C\C/C=C\C/C=C\C/C=C\C/C=C\C/C=C\CCC(=O)NCCNC(=O)/C=C/C(=O)OC]

(E)-methyl 4-(2-(4Z,7Z,10Z,13Z,16Z,19Z)-docosa-4,7,10,13,16,19-hexaenamidoethylamino)-4-oxobut-2-enoate or a pharmaceutically acceptable salt thereof and (b) a statin and one or more pharmaceutically acceptable excipients.

In one embodiment,

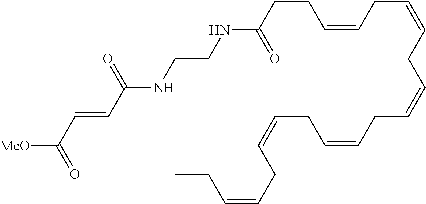 [SMILES: CC/C=C\C/C=C\C/C=C\C/C=C\C/C=C\C/C=C\CCC(=O)NCCNC(=O)/C=C/C(=O)OC]

(E)-methyl 4-(2-(4Z,7Z,10Z,13Z,16Z,19Z)-docosa-4,7,10,13,16,19-hexaenamidoethylamino)-4-oxobut-2-enoate or a pharmaceutically acceptable salt thereof at a dose range of about 20 mg to about 5000 mg of the FAFD per day and the statin at a dose range of 10 mg to 80 mg.

In certain embodiments, FAFDs provided by the present disclosure and pharmaceutical compositions thereof may be administered to a subject for treating heart failure with preserved ejection fraction (HFPEF) in combination with an aldosterone antagonist.

In certain embodiments, FAFDs provided by the present disclosure and pharmaceutical compositions thereof may be administered to a subject for treating heart failure with preserved ejection fraction (HFPEF) in combination with an angiotensin-receptor neprilysin inhibitor (ARNI).

In certain embodiments, FAFDs provided by the present disclosure and pharmaceutical compositions thereof may be administered to a subject for treating heart failure with reduced ejection fraction (HFREF) in combination with a therapy or another therapeutic agent known or believed to be effective in treating HFREF. Useful drugs for treating HFREF include antitensin-modulating agents, diuretics such as furosemide, bumetanie, hydrochlorothiazide, chlorthalidone, chlorthiazide, spironolactone, eplerenone: beta blockers such as bisoprolol, carvedilol, and metroprolol; positive inotropes such as digoxin, milrinone, and dobutamine; alternative vasodilators such as isosorbide dinitrate/hydralazine; aldosterone receptor antagonists; recombinant neuroendocrine hormones such as nesiritide; angiotensin receptor-neprilysin inhibitors such as LCZ696; and vasopressin receptor antagonists such as tolvaptan and conivaptan.

### Equivalents

Those skilled in the art will recognize, or be able to ascertain, using no more than routine experimentation, numerous equivalents to the specific embodiments described specifically herein. Such equivalents are intended to be encompassed in the scope of the following claims.

### PROPHETIC EXAMPLE 1

The following prophetic example serves to provide approximate dosage levels of FAFDs to achieve the intended effect, for example treatment of heart failure with preserved ejection fraction (HFPEF). Based on the literature, a few assumptions about the dosage can be made, as will be described in further detail below.

Docosahexaenoic acid (DHA) or pharmaceutically acceptable salts, esters, amides, epoxides, and prodrugs thereof have been used by Timothy O'Connell et. al. for treating or limiting development of heart failure with preserved ejection fraction (HFPEF). The pharmaceutical composition comprises purified or synthesized DHA or a pharmaceutically acceptable salt, ester, amide, epoxide, or prodrug thereof. In certain other embodiments, DHA, or a pharmaceutically acceptable salt, ester, amide, epoxide, or prodrug thereof, is administered to the subject at a concentration from about 5 mg/kg of body weight/day to about 50 mg/kg of body weight/day. In certain other embodiments, the dose is about 600 mg/day to about 1000 mg/day of DHA, or in certain other embodiments, about 800 mg/day.

The full mechanism of fumaric acid esters such as dimethyl fumarate (DMF) and its primary metabolite, monomethyl fumarate (MMF), is not completely understood, but their beneficial effects appear to be mediated, at least in part, through the activation of the NRF2 antioxidant response pathway, which further increases expression of ARE, which increases expression of detoxifying enzymes and antioxidant proteins. For example, a recent publication showed, using ribonucleic acid (RNA) and protein, that CAT-4001, a MMF, inihibits NFkB and activates Nrf2 in vitro and in patient cells (Milne J, et al., “Catabasis Investor Day,” November 17, 2016, New York City, pp. 39-40, the disclosure of which is incorporated by reference in its entirety).

Further, NRF2 deficiency, demonstrated by NRF2 knockout in murine models, results in an earlier onset of cardiac dysfunction induced by pressure and volume overload (Li et al Arterioscler Thromb Vase Biol. 2009, 29(11), 1843-50). Certain NRF2 activators such as sulforaphane, curcumin, carbobenzoxy-Leu-Leu (MG132), resveratrol, garlic organosulfur compounds, allicin, 4-hydroxynonenal (4-HNE), α-lipoic acid, hydrogen sulfate, and 17α-estradiol have been used as therapeutic targets to reduce cardiac remodeling, but prodrugs of monomethyl fumarate/ FAFDs have not been used yet to reduce cardiac remodeling (Zhou et al; J Appl Physiol. 2015,119(8), 944-951).

Fumarates are cardioprotective in acute situations via activation of the NRF2 pathway in acute ischemia due to myocardial infarction (Ashrafian et al; Cell Metab. 2012, 15(3), 361-71). However, Ashrafian et. al claims that fumarates are harmful in chronic situations, including heart failure. FAFDs (molecular conjugate of fumaric acid and fatty acid) are herein proposed to achieve the intended effect, for example, treatment of chronic heart failure with preserved ejection fraction (HFPEF).

Dimethyl Fumarate has been tested for multiple sclerosis and psoriasis at multiple dosages in the past, including 120 mg, 240 mg, daily, BID, and TID. The side effect profile was similar regardless of which dosage was used.

Further, in order to determine dosage of a FAFD, a dose escalation study may be conducted to find a comparable dosage of the FAFD to DMF's 240 mg dose, by comparing plasma levels of MMF. For example, one FAFDs known as (I-1), a compound having formula:

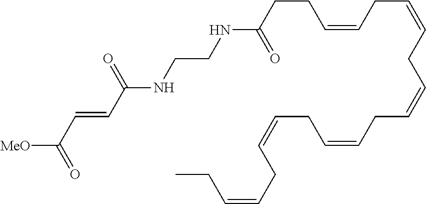 [SMILES: CC/C=C\C/C=C\C/C=C\C/C=C\C/C=C\C/C=C\CCC(=O)NCCNC(=O)/C=C/C(=O)OC]

will be tested to find a comparable dose of 240 mg DMF (Tecfidera) by comparing plasma levels of MMF. Various dosages of a FAFD will be tested in HFPEF subjects so that the dosage that is comparable to a DMF dosage of 120 mg, 240 mg, daily, BID, and TID may be determined more precisely. Using (I-1) as one such FAFD, such dosage is likely to be calculated as near about 20 mg to about 5000 mg of the FAFD per day. In some embodiments, such dosage may contain about 20, 50, 75, 100, 150, 250, 500, 750, 1000, 1250, 2500, 3500, or 5000 mg of the FAFD. In one embodiment, the composition is in the form of a tablet that can be scored. Effective plasma levels of the FAFD can range from about 0.002 mg to about 100 mg per kg of body weight per day.

Furthermore, pro-inflammatory cytokines IL-6 and TNF-α are raised in HFPEF, which may lead to increase activity of VCAM, E-Selection, and NADPH oxidase, which increase ROS in coronary microvasculature endothelial cells, leading to the hallmarks of HFPEF: ventricular stiffness, impaired relaxation, and cardiac dysfunction. The FAFD may reduce damage of ROS in heart failure by multiple pathways including increasing the NRF2/ARE pathway, and possibly by reducing NF-kB, which reduces IL-6 and TNF-α.

LCZ696, a combined angiotensin receptor neprilysin inhibitor (ARNI) that has recently shown to reduce mortality in HFREF but not in HFPEF subjects. LCZ696 inhibits natriuretic peptide breakdown and enhances cGMP activation, and in HFPEF was associated with incremental reductions in circulating N-terminal pro-B-type natriuretic peptide (NT-proBNP) levels when compared to treatment with the ARB valsartan, alone. However, these reductions were incremental, and it is yet to be seen whether LCZ696 or other angiotensin receptor-neprilysin inhibitors will lead to any significant mortality or clinical benefit in HFPEF subjects. Furthermore, the comparison with ARB valsartan alone, is flawed in that ARB valsartan is used in the treatment of HFREF but not in HFPEF.

The subjects' baseline TNF-alpha, IL-6, NT-proBNP will be measured at the start of the trial and compared to levels at various intervals (weeks to months to years) to determine the ideal dosage based on reductions in TNF-alpha, IL-6, and/or NT-proBNP. Such a dosage will then be tested in a larger group of HFPEF subjects to measure changes in morbidity and mortality. Thus, an ideal dosage of a FAFD for treating HFPEF will be comparable to a dosage of 120 mg or 240 mg, daily, BID, or TID of DMF (Tecfidera), by measuring MMF concentrations in the blood. In the case of compound (I-1), this dosage range may likely be near about 20 mg to about 5000 mg of the FAFD per day, however, the exact dosage will be determined in subjects based on the description above.

### PROPHETIC EXAMPLE 2

Based on the above prophetic example, an exemplary, non-limiting embodiment is described in detail below. As described herein, a user may include a male or female between the ages of 50 to 100 with ejection fraction of greater than 40%, and more likely to be a female with a documented history of high blood pressure, obesity, diabetes, renal disease and/or COPD, with at least one episode of fluid overload, or who has HFPEF or is at risk of developing (HFPEF).

The most common disease leading to HFPEF is systolic hypertension, which is present in more than 85% of subjects. Subjects with HFPEF have normal left ventricular (LV) end-diastolic volume and normal (or near-normal) EF and stroke volume and commonly exhibit concentric remodeling of either the LV chamber and/or cardiomyocytes.

Subjects with HFPEF have a devastating 5-year mortality rate (approaching 60%), costly morbidity (6-month hospitalization rate of 50%), and debilitating symptoms (maximum myocardial oxygen consumption [MVo₂] averaging 14 mL/g/min).

More than half of heart failure subjects have heart failure with preserved ejection fraction (HFPEF). Morbidity and mortality of HFPEF are similar to HFREF; however, medications proven effective in HFREF have not been found to be effective in HFPEF. At present, there are no approved treatments to reduce mortality in HFPEF. In HFREF, medications such as beta-blockers, ace-inhibitors, angiotensin receptor blockers, isosorbide dinitrate, hydralazine, aldosterone inhibitors, and angiotensin receptor neprilysin inhibitors have been shown to provide benefit. However, these medications have not shown to be beneficial in subjects with HFPEF, and are not approved therapies for HFPEF.

### PROPHETIC EXAMPLE 3

The following prophetic example serves to provide a combination therapy for subjects with HFPEF, which includes a FAFD with a statin. To date there has been no prospective studies of statins in subjects with HFPEF. However, statins have pleotropic effects, in which they have been shown to be beneficial to non-HFPEF subjects beyond what was predicted based on their ability to reduce cholesterol, likely through anti-inflammatory pathways. By combining a statin with a FAFD, a synergistic effect to reduce the ROS associated with HFPEF is expected, which in turn will reduce stiffness in HFPEF and also reduce biomarkers such as IL-6, TNF-alpha, or NT-proBNP, and ultimately improve survival in HFPEF subjects. In one such example, a dose range between about 20 mg to about 5000 mg of the FAFD (I-1) is given to a subject with a statin dosage between 10 mg to 80 mg.

As used in the description and claims, the singular form “a”, “an” and “the” include both singular and plural references unless the context clearly dictates otherwise. For example, the term “prodrug” may include, and is contemplated to include, a plurality of prodrugs. At times, the claims and disclosure may include terms such as “a plurality,” “one or more,” or “at least one;” however, the absence of such terms is not intended to mean, and should not be interpreted to mean, that a plurality is not conceived.

The term “about” or “approximately,” when used before a numerical designation or range (e.g., to define a length or pressure), indicates approximations which may vary by (+) or (−) 5%, 1% or 0.1%. All numerical ranges provided herein are inclusive of the stated start and end numbers. The term “substantially” indicates mostly (i.e., greater than 50%) or essentially all of a method, substance, or composition.

As used herein, the term “comprising” or “comprises” is intended to mean that the methods and compositions include the recited elements, and may additionally include any other elements. “Consisting essentially of” shall mean that the methods and compositions include the recited elements and exclude other elements of essential significance to the combination for the stated purpose. Thus, a method or composition consisting essentially of the elements as defined herein would not exclude other materials, features, or steps that do not materially affect the basic and novel characteristic(s) of the claimed disclosure. “Consisting of” shall mean that the methods and compositions include the recited elements and exclude anything more than a trivial or inconsequential element or step. Embodiments defined by each of these transitional terms are within the scope of this disclosure.

The terms “optionally” as used herein means that a subsequently described event or circumstance may but need not occur, and that the description includes instances where the event or circumstance occurs and instances in which it does not.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed:

1. A method of treating a heart failure disease in a subject in need thereof, the method comprising administering to the subject a therapeutically effective amount of one or more fatty acid fumarate derivatives of Formula I 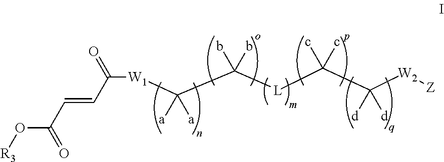
or a pharmaceutically acceptable salt, hydrate, enantiomer, or stereoisomer thereof; wherein
each W1 and W2 is independently null, O, S, NH, or NR, or W1 and W2 can be taken together to form an optionally substituted imidazolidine or piperazine group; each a, b, c, and d, is independently —H, —D, —CH3, —OCH3, —OCH2CH3, —C(O)OR, —O—Z, or benzyl, or two of a, b, c, and d can be taken together, along with the single carbon to which they are bound, to form a cycloalkyl or heterocycle; each n, o, p, and q is independently 0,1, or 2;
each L is independently null, —O—, —C(O)—, —S—, —S(O)—, —S(O)2—, —S—S—, —(C1-C6 alkyl)—, —(C3-C6 cycloalkyl)-, a heterocycle, a heteroaryl, 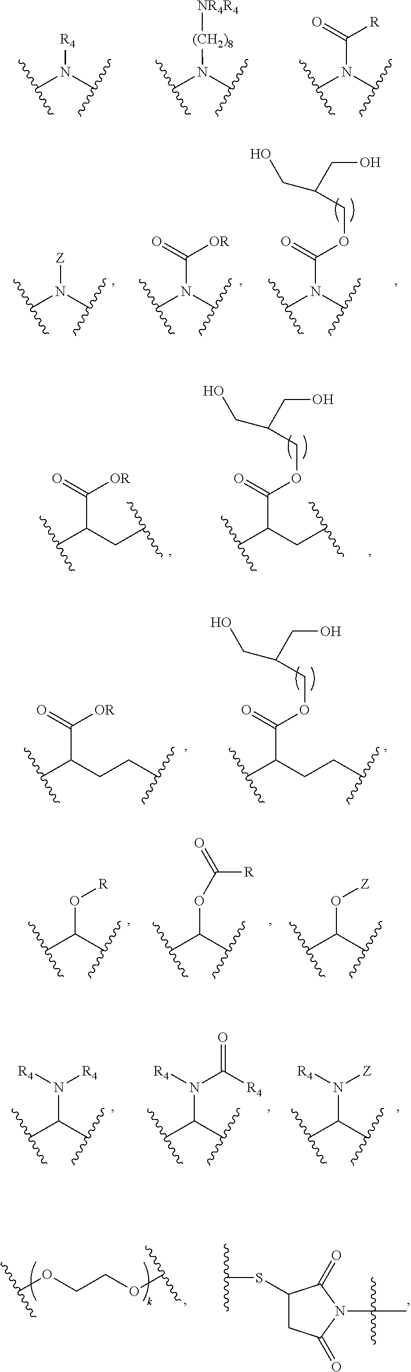 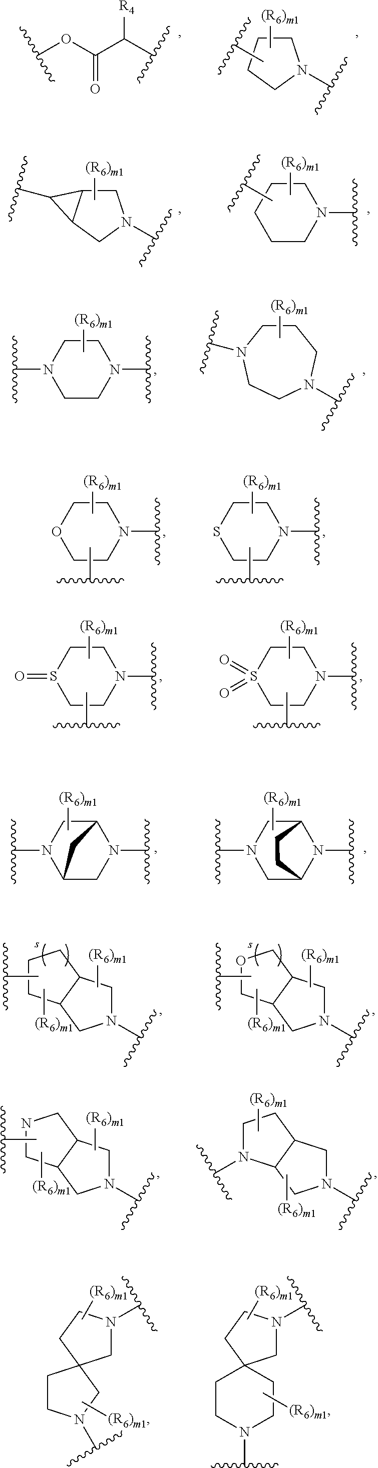 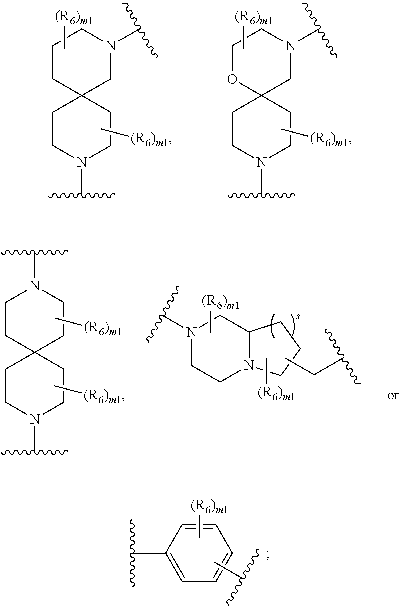
wherein the representation of L is not limited directionally left to right as is depicted, rather either the left side or the right side of L can be bound to the W₁ side of the compound of Formula I;
each R₆ is independently —H, —D, —C₁-C₄ alkyl, -halogen, cyano, oxo, thiooxo, —OH, —C(O)C₁-C₄ alkyl, —O-aryl, —O-benzyl, —OC(O)C₁-C₄ alkyl, —C₂-C₃ alkene, —C₂-C₃ alkyne, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, or —S(O)₂C₁-C₃ alkyl;
each g is independently 2, 3, or 4;
each h is independently 1, 2, 3, or 4;
each m is independently 0, 1, 2, or 3; if m is more than 1, then L can be the same or different;
each ml is independently 0, 1, 2, or 3;
k is 0, 1, 2, or 3;
z is 1, 2, or 3;
each R₄ is independently H or optionally substituted C₁-C₆ alkyl, wherein a methylene unit of the C₁-C₆ alkyl can be optionally substituted for either O or NR, and in NR₄R_{4,} both R₄ when taken together with the nitrogen to which they are attached can form a heterocyclic ring such as a pyrrolidine, piperidine, morpholine, piperazine or pyrrole; each Z is independently H, 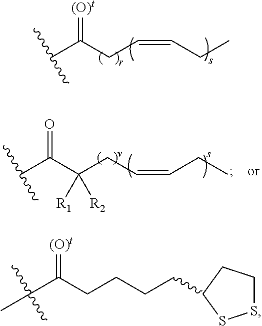
provided that
there is at least one 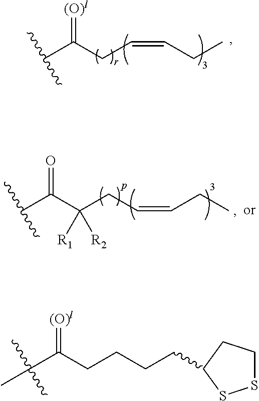
in the compound;
each t is independently 0 or 1;
each r is independently 2,3, or 7;
each s is independently 3, 5, or 6;
each v is independently 1,2, or 6;
each R₁ and R₂ is independently —H, —D, —C₁-C₄ alkyl, -halogen, —OH, —C(O)C₁-C₄ alkyl, —O-aryl, —O-benzyl, —OC(O)C₁-C₄ alkyl, —C₂-C₃ alkene, —C₂-C₃ alkyne, —NH₂, —NH(C₁-C₃ alkyl), —N(C₁-C₃ alkyl)₂, —NH(C(O)C₁-C₃ alkyl), —N(C(O)C₁-C₃ alkyl)₂, —SH, —S(C₁-C₃ alkyl), —S(O)C₁-C₃ alkyl, or —S(O)₂C₁-C₃ alkyl;
each R₃ is independently H, or —C₁-C₆ alkyl;
each R₅ is independently e, H, or straight or branched C₁-C₁₀ alkyl which can be optionally substituted with OH, NH₂, CO₂R, CONH₂, phenyl, C₆H₄OH, imidazole or arginine;
each e is independently H or any one of the side chains of the naturally occurring amino acids;
each R is independently —H, —C₁-C₃ alkyl, or straight or branched C₁-C₄ alkyl optionally substituted with OH, or halogen;
provided that
when each of m, n, o, p, and q is 0, W₁ and W₂ are each null, and Z is 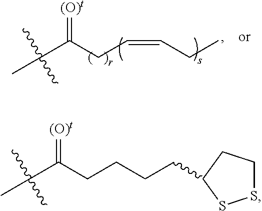
then t must be 0; and
when each of m, n, o, p, and q is 0, and W₁ and W₂ are each null, then Z must not be

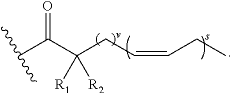

2. The method of claim 1, wherein the heart failure disease is one of: heart failure with preserved ejection fraction (HFPEF); heart failure with ejection fraction ≧40%; diastolic heart failure; heart failure with elevated levels of TNF-α, IL-6, CRP, or TGF-β; hypertension with risk of developing HFPEF; atrial fibrillation with risk of developing HFPEF; diabetes with risk of developing HFPEF; COPD with risk of developing HFPEF; ischemic heart disease with risk of developing HFPEF; obesity with risk of developing HFPEF; chronic heart failure; compensated heart failure; and decompensated heart failure.

3. The method of claim 2, wherein the heart failure disease is heart failure with preserved ejection fraction (HFPEF).

4. The method of claim 1, wherein the fatty acid fumarate derivative is a compound of formula 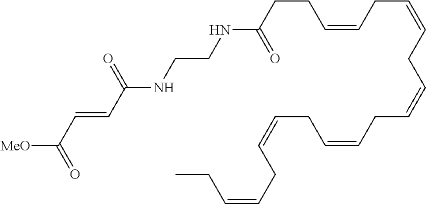 [SMILES: CC/C=C\C/C=C\C/C=C\C/C=C\C/C=C\C/C=C\CCC(=O)NCCNC(=O)/C=C/C(=O)OC]
(E)-methyl 4-(2-(4Z,7Z,10Z,13Z,16Z,19Z)-docosa-4,7,10,13,16,19-hexaenamidoethy lamino)-4-oxobut-2-enoate (I-1) or a pharmaceutically acceptable salt thereof.

5. The method of claim 1, wherein a pharmaceutical composition is administered to the subject, wherein said pharmaceutical composition comprises a therapeutically effective amount of 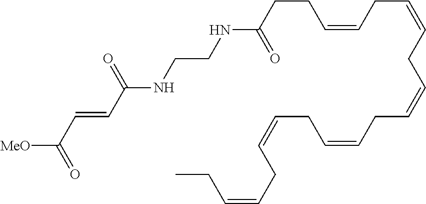 [SMILES: CC/C=C\C/C=C\C/C=C\C/C=C\C/C=C\C/C=C\CCC(=O)NCCNC(=O)/C=C/C(=O)OC]
(E)-methyl 4-(2-(4Z,7Z,10Z,13Z,16Z,19Z)-docosa-4,7,10,13,16,19-hexaenamidoethylamino)-4-oxobut-2-enoate (I-1) or a pharmaceutically acceptable salt thereof that is shown to provide MMF plasma exposure comparable to dimethyl fumarate (DMF) 120 mg to 720 mg per day.

6. The method of claim 1, wherein a pharmaceutical composition is administered to the subject, wherein said pharmaceutical composition comprises about 20 mg to about 5000 mg of 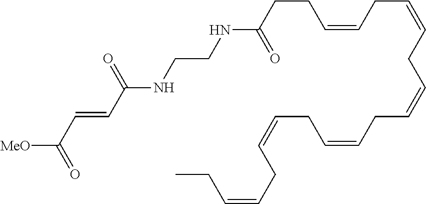 [SMILES: CC/C=C\C/C=C\C/C=C\C/C=C\C/C=C\C/C=C\CCC(=O)NCCNC(=O)/C=C/C(=O)OC]
(E)-methyl 4-(2-(4Z,7Z,10Z,13Z,16Z,19Z)-docosa-4,7,10,13,16,19-hexaenamidoethy lamino)-4-oxobut-2-enoate or a pharmaceutically acceptable salt thereof.

7. The method of claim 1, wherein fatty acid fumarate derivative of formula 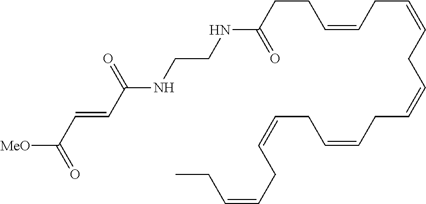 [SMILES: CC/C=C\C/C=C\C/C=C\C/C=C\C/C=C\C/C=C\CCC(=O)NCCNC(=O)/C=C/C(=O)OC]
(E)-methyl 4-(2-(4Z,7Z,10Z,13Z,16Z,19Z)-docosa-4,7,10,13,16,19-hexaenamidoethylamino)-4-oxobut-2-enoate or a pharmaceutically acceptable salt thereof is administered in combination with one or more second agents useful for treating heart failure.

8. The method of claim 7, wherein the second agent is selected from the group consisting of: a diuretic, an ace-inhibitor, a beta-blocker, an angiotensin receptor blocker, isosorbide dinitrate, hydralazine, an angiotensin receptor-neprilysin inhibitor, an aldosterone antagonist, a PDE5 inhibitor, a statin, a neprilysin inhibitor, an aldosterone inhibitor, and an antitumor necrosis factor-alpha therapy.

9. The method of claim 8, wherein the second agent is the statin.

10. A pharmaceutical composition comprising (a) 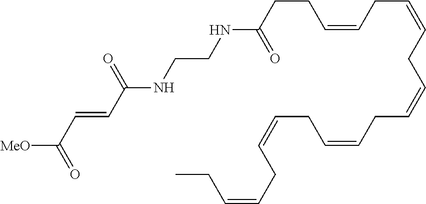 [SMILES: CC/C=C\C/C=C\C/C=C\C/C=C\C/C=C\C/C=C\CCC(=O)NCCNC(=O)/C=C/C(=O)OC]
(E)-methyl 4-(2-(4Z,7Z,10Z,13Z,16Z,19Z)-docosa-4,7,10,13,16,19-hexaenamidoethylamino)-4-oxobut-2-enoate or a pharmaceutically acceptable salt thereof and (b) a statin and one or more pharmaceutically acceptable excipients.

11. The pharmaceutical composition of claim 10, 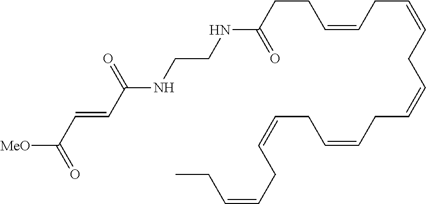 [SMILES: CC/C=C\C/C=C\C/C=C\C/C=C\C/C=C\C/C=C\CCC(=O)NCCNC(=O)/C=C/C(=O)OC]
(E)-methyl 4-(2-(4Z,7Z,10Z,13Z,16Z,19Z)-docosa-4,7,10,13,16,19-hexaenamidoethylamino)-4-oxobut-2-enoate or a pharmaceutically acceptable salt thereof at a dose range of 20 mg to about 5000 mg and the statin at a dose range of 10 mg to 80 mg.

12. The pharmaceutical composition of claim 10, wherein statin is selected from group consisting of atorvastatin, fluvastatin, lovastatin, pravastatin, rosuvastatin, and simvastatin.

13. A method of treating a heart failure disease in a subject in need thereof, the method comprising: administering to the subject a therapeutically effective amount of (a) ( 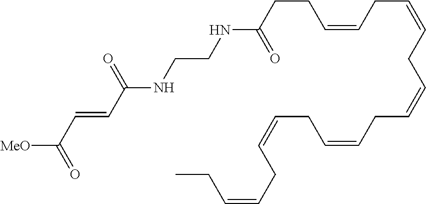 [SMILES: CC/C=C\C/C=C\C/C=C\C/C=C\C/C=C\C/C=C\CCC(=O)NCCNC(=O)/C=C/C(=O)OC]
(E)-methyl 4-(2-(4Z,7Z,10Z,13Z,16Z,19Z)-docosa-4,7,10,13,16,19-hexaenamidoethylamino)-4-oxobut-2-enoate or a pharmaceutically acceptable salt thereof and either separately or together with (b) a statin.

14. The method of claim 13, wherein the heart failure disease is heart failure with preserved ejection fraction (HFPEF).
